(19) United States
(12) Patent Application Publication
(10) Pub. No.: US 2019/0298193 A1
(43) Pub. Date: Oct. 3, 2019

(54) LOW PRESSURE ACTUATION BLOOD PRESSURE MONITORING

(71) Applicant: Angilytics Inc., Berkeley, CA (US)

(72) Inventors: Xiaoding Zhuo KRAUSE, Berkeley, CA (US); Piotr PRZYBYSZEWSKI, San Jose, CA (US)

(21) Appl. No.: 16/437,430

(22) Filed: Jun. 11, 2019

Related U.S. Application Data
(63) Continuation-in-part of application No. 15/424,608, filed on Feb. 3, 2017.
(60) Provisional application No. 62/711,530, filed on Jul. 29, 2018.
(60) Provisional application No. 62/341,601, filed on May 25, 2016.
(60) Provisional application No. 62/290,642, filed on Feb. 3, 2016.

(51) Int. Cl. A61B 5/022; A61B 5/021

(52) U.S. Cl. CPC A61B 5/02225; A61B 5/0205; A61B 5/02116

(57) ABSTRACT

In one embodiment, a system comprises a band; a pressure actuator to apply external pressure through the band to a part of a human body; circuitry to control the pressure actuator to apply the external pressure changing only in a pressure range less than 80 mmHg in a measurement mode; a pressure sensor to sense, from the band, a waveform signal responsive to an application of the external pressure by the pressure actuator in the measurement mode, wherein the waveform signal is indicative of a pressure response of arterial pressure; a pulse waveform velocity (PWV) sensor to sense one or more signals associated with PWV; and a processor to calculate a blood pressure value based on the waveform signal from the pressure sensor and the signal(s) associated with PWV.

### CROSS REFERENCE TO RELATED APPLICATIONS

This application claims priority to U.S. Provisional Application No. 62/711,530, entitled “System for Non-Invasive, Non-Occlusive Blood Pressure Monitoring Based on: Sensor Fusion, Cardiovascular Models, and Machine Learning” and filed on Jul. 29, 2018, which is incorporated by reference herein. This application is also a continuation in part of U.S. patent application Ser. No. 15/424,608, entitled “Non-invasive and non-occlusive blood pressure monitoring devices and methods” and filed on Feb. 3, 2017, which claims priority to Provisional U.S. Patent Application 62/341,601, entitled “Personalized non-invasive, non-occlusive blood pressure measurement using oscillometric waveform analysis” and filed on May 25, 2016 and Provisional U.S. Patent Application 62/290,642, entitled “Continuous non-invasive, non-occlusive blood pressure measurement using subject-specific and demographic-specific pulse pressure models and filed on Feb. 3, 2016, each of which is incorporated by reference herein.

### TECHNICAL FIELD

The present disclosure relates to blood pressure monitoring.

### BACKGROUND

Most non-invasive blood pressure monitors available today rely on oscillometric method, which involves the application of an external pressure surrounding a segment of the artery (either upper arm or the wrist) and measures the pressure waveforms (oscillations) as the external pressure changes from above the systolic pressure to below the diastolic pressure or vice versa. This requires a powerful pressure actuation system that enables pressure change from 0 mmHg to as high as 300 mmHg in order to meet the application needs. The method has a solid theoretical base in models derived from physics and physiology, which gives it full potential to meet accuracy requirements set in the FDA recognized standards. However, this high cuff pressure requirement blocks blood circulation and is painful for hypertensive patients or repeated measurements. There is an unmet need for a non-invasive, cuff-less, calibration-less, comfortable 24-hour blood pressure monitor.

A common approach to this problem involves long-researched method based on measurement of the Pulse Wave Velocity (PWV). However, conclusions from experiments and theoretical research show insufficiency of using the PWV alone to calculate blood pressure, especially due to the changing vascular tone and smooth muscle regulation. As a result, PWV based systems have to be calibrated on a regular basis in order to achieve accurate results.

### BRIEF DESCRIPTION OF THE DRAWINGS

The included drawings are for illustrative purposes and serve to provide examples of possible structures and operations for the disclosed inventive systems, apparatus, methods and computer-readable storage media. These drawings in no way limit any changes in form and detail that may be made by one skilled in the art without departing from the spirit and scope of the disclosed implementations.

FIG. 1A is a graph illustrating systolic and diastolic arterial volumes as functions of the external pressure, and FIG. 1B is a graph illustrating the Oscillometric Waveform Magnitude (OWM, defined as the peak height of the oscillometric envelop) as functions of the external pressure. The low pressure actuation method operates at the Zone I of the diagram.

FIG. 2 is a flowchart showing blood pressure calculation in an oscillometric method.

FIG. 3A is a diagram illustrating steps for accurate inference of blood pressure using the low pressure actuation method with Pulse Wave Velocity input, and FIG. 3B is a diagram illustrating steps for accurate inference of blood pressure using the low pressure actuation method with a one-time-per patient calibration.

FIG. 4A is a graph illustrating Log-OWM versus the external pressure (e.g., similar to FIG. 1B with logarithmic vertical axis), and FIG. 4B shows sample data for Zone I from FIG. 4A, the pressure range of the operation for the low pressure actuation method. There is a fitted linear relationship between the Log-OWM and external pressure in Zone I.

FIG. 5 is a flowchart illustrating different ways of determining PWV_{din} from input sensors.

FIG. 6 is a flowchart illustrating calculation of a blood pressure value using the low-pressure oscillometric waveforms in Zone I and the PWV.

FIG. 7 is a flowchart illustrating calculation of a blood pressure value in extended low-pressure actuation method, based from oscillometric waveforms from both Zone I and Zone II.

FIG. 8 is a flowchart illustrating calculation of parameters describing cardiovascular compliance.

FIG. 9 is a flowchart illustrating calculation of a blood pressure value based on low-pressure oscillometry and known value of Vₐ₀ (either from earlier scans or from one-time-per-patient calibration).

FIG. 10 is a flowchart illustrating calculation of a blood pressure value during an external one-time-per-user calibration (quantities P_{a.dia} and P_{a.sys} may be measured with an external reference system).

FIG. 11 is a flowchart illustrating calculation of a blood pressure value during an internal one-time-per-user calibration (quantities P_{a.dia} and P_{a.sys} may be measured with the same system by extending the pressure range to above the diastolic pressure to include Zone II or Zone II and III).

FIG. 12A illustrates machine learning from features for blood pressure value determinations, and FIG. 12B illustrates deep learning for blood pressure value determinations.

FIG. 13 is a table showing demographic statistics of 150 subjects participated in the validation study.

FIG. 14 is a graph including validation data from test subjects that demonstrates high accuracy for both systolic and diastolic pressures.

FIG. 15 is a block diagram illustrating one embodiment of a comfortable, non-occlusive blood pressure monitoring device in which the device is a 24/7 wristband.

FIG. 16 is a block diagram of another embodiment of a comfortable, non-occlusive blood pressure monitoring device in which the device is a 24/7 wristband.

FIG. 17 is a block diagram of a low pressure actuation blood pressure monitoring system including a wearable device, according to various embodiments.

FIG. 18 is a block diagram of a low pressure actuation blood pressure monitoring system in which the wearable device includes a motion sensor, according to various embodiments.

FIG. 19 is a block diagram of a low pressure actuation blood pressure monitoring system in which the wearable device wirelessly transmits raw measurement data or information derived therefrom to a cloud device for remote calculation of blood pressure value, according to various embodiments.

FIG. 20 is a block diagram of a low pressure actuation blood pressure monitoring system in which the wearable device transmits raw measurement data or information derived therefrom over a short range wireless connection or a wire to a portable device for calculation of the blood pressure value by the portable device.

FIG. 21 illustrates blood pressure models that may be utilized by any processor described herein to calculate a blood pressure value based on a waveform signal from a pressure sensor and signals associated with PWV, according to various embodiments.

FIG. 22A illustrates a process for calculating a blood pressure value from reduced OWM data collection frequency, signals associated with PWV, and longitudinal data of the population, according to various embodiments.

FIG. 22B illustrates a process for calculating continuous blood pressure using information about the OWM data (FIG. 22A), continuous PWV signal, and the longitudinal data of the user, according to various embodiments.

FIG. 22C illustrates a process for calculating continuous blood pressure using continuous PWV signal and inferences of the information about the OWM from a large sample of the longitudinal data of population, according to various embodiments.

FIG. 23 illustrates a wearable device used in any low pressure actuation blood pressure monitoring system described herein, according to various embodiments.

FIG. 24 illustrates a blood pressure monitoring system in which a feedback is used to help with blood pressure management, and update a model used by the wearable device to calculate the blood pressure value, according to various embodiments.

FIG. 25 illustrates blood pressure waveforms and other information that may be generated or calculated by any processor described herein, according to various embodiments.

### DETAILED DESCRIPTION

Some embodiments of the current innovation are discussed in detail below. In describing embodiments, specific terminology is employed for the sake of clarity. However, the invention is not intended to be limited to the specific terminology so selected. Embodiments are examples of the innovations and do not limit the innovations. A person skilled in the relevant art will recognize that other equivalent components can be employed, and other methods developed without departing from the broad concepts of the current innovations.

### Overview

According to the American Heart Association, in January of 2018, one hundred million people in the U.S. alone have been suffering from hypertension, or high blood pressure, and this number is growing exponentially year-over-year. Hypertension is a leading sign of risk for potentially fatal cardiac diseases, such as heart attack, stroke, and coronary heart diseases, etc. (https://www.heart.org/en/news/2018/05/01/more-than-100-million-americans-have-high-blood-pressure-aha-says). However, despite the large number of people at risk of and suffering from hypertension-related diseases, it is a very difficult condition to monitor and manage successfully to prevent escalation in most patients, primarily due to the blood pressure devices on the current market only enables point-of-care management. There is a need to address this problem with a new blood pressure monitoring device and method that is not only comfortable and convenient for frequent measurement, but also more patient and doctor friendly, more representative of a patient's blood pressure condition, and ultimately, more successful in helping doctors with monitoring and identifying signs associated with more serious cardiovascular conditions, and therefore preventing hypertension-related diseases and deaths.

It is observed herein that lack of effective monitoring of blood pressure of potentially high-risk hypertension patients over an extended period of time is currently a major issue within the medical industry, making it costly in terms of medical expenses for both the patients and hospitals. It also degrades the patients' overall health, as signs of more serious disease are missed until it is too late to treat. The key reason for the ineffectiveness is that there is no accurate device comfortable and convenient enough for frequent measurement and long-term monitoring. Doctors do not receive representative data of the patient's blood pressure variability to successfully monitor and treat diseases.

The low pressure actuation invention reduces the operating range of external pressure to a level comfortable enough for frequent, 24 hour measurement, and enables the reduction of the footprint of hardware so that it is highly portable. Some blood pressure monitoring methods described herein use oscillometric waveforms generated from the application of low range external pressure and measurements of pulse wave velocity (PWV) to calculate blood pressure based on at least two data points of oscillometric waveforms. The external pressure range is less than 80 mmHg (below a diastolic pressure), 20-50 mmHg, or 20-40 mmHg, depending on the integration and design of hardware. The result is accurate, does not require calibration or recalibration, and external pressure is non-occlusive, so it is possible to monitor blood pressure frequently and comfortably. In other words, the method calculates blood pressure based on the pressure response of external pressure in the low range as a response of time. The method does NOT use uncomfortable external pressure (Over 80, 50, or 40 mmHg) during the measuring process. PWV is measured by signals from two photoplethysmography's (PPG), or one PPG and one electrocardiograph (ECG), or two PPGs and one ECG.

Some blood pressure measuring systems and devices described herein may include a band, a pressure actuator operable to apply external pressure through the band to a part of a human body, a pressure sensor operable to sense from the band, a waveform signal related to a pressure response of arterial pressure, a PWV sensor operable to sense signals related to pulse wave velocity (PWV), a controller which has a first measurement mode operable to control the pressure actuator to apply the external pressure changing only in a certain range less than 80 mmHg, and a calculator operable to receive the waveform signal from the pressure sensor (oscillometric waveform), the signal from the PWV sensor(s) (PWV signal), and calculate a blood pressure based on the oscillometric waveform and the PWV signal in the first mode. In this mode, external pressure is only in a low range, much less than the highest pressure required by traditional oscillometric methods. Therefore, it is comfortable for the user for both long term and short term use. The device comprising; the band, the pressure actuator, the pressure sensor, the PWV sensor(s), the controller, and the transmitter to transmit the oscillometric waveform and the PWV signal to the calculator. The calculation does not need to be done in the device and can be done in a separate system, for example, in another device or in the cloud.

In some embodiments, the operating range of external pressure is less than 50 mmHg or 40 mmHg.

In some embodiments, the operating range of external pressure is at least 20 mmHg.

In some embodiments, other than the first mode, the controller has a second mode operable to control the pressure actuator to apply the external pressure changing beyond the said operating range of external pressure. For example, in the second mode, the device works as a sphygmomanometer once in a while to calibrate the calculation. Because it is uncomfortable, it is better to limit the use of the second mode only in suitable situations. Another example is that the device operates in a pressure range in parts of both Zone I and Zone II, and inferences blood pressure with the additional information of parts or all of Zone II. A third example is the use of a standalone device for one-time calibration, and the derived subject-specific parameters are feed into the model for blood pressure inference.

In some embodiments, the controller has a third mode operable to control the calculator to calculate a blood pressure based on the signal from the PWV sensor(s) and control the pressure actuator not to work. The third mode of operation is when the state of the person is relatively stable and the controller decides that for a certain period of time, blood pressure measurement can be done solely from the PWV sensor without the application of pressure.

In some embodiments, the device further comprising a motion sensor operable to detect the motion of the user, and the controller to prohibit operation when the motion is greater than a certain threshold. This allows the controller to determine the best time to take measurements to minimize the impact of motion artifacts.

In some embodiments, the device further comprising a motion sensor operable to detect the motion of the user, and the controller to prohibit the second mode when the motion is less than a certain threshold.

This avoids frequent usage of the second mode. It also minimizes the application of a pressure beyond Zone I to ensure comfortable measurements for long-term monitoring.

In some embodiments, the controller prohibits the first mode and works in the third mode when the motion is less than a certain threshold. The device can determine the time for the third mode of operation when the user wearing the device is sleeping or in a state that has little change on the cardiovascular parameters.

In some embodiments, the device further comprising a command receiver operable to receive a command to start pressure actuation, and the controller starting the pressure actuation when the receiver receives the command. This avoids frequent usage of pressure actuation and uses it only when needed for the accuracy of measurement.

In some embodiments, the device further comprising a command receiver operable to receive a command to start the second mode, and the controller starting the second mode when the receiver receives the command. This avoids frequent usage of the second mode. The user or doctor can start the second mode only when needed.

In some embodiments, the PWV sensor(s) comprising: two photoplethysmography's (PPG), one PPG and one electrocardiograph (ECG), two PPGs and one ECG, one or two sets of a pressure actuator plus a pressure sensor in place of PPGs in the previous 3 options, or Doppler radar.

In some embodiments, the system further comprising a recorder operable to record the blood pressure on the device, in the cloud or both.

In some embodiments, the recorder is operable to record the (series of) blood pressures with time information of the measurements. The user or doctor can check any changes of the condition of the blood pressure or user, and use statistical assessment such as maximum, minimum, mean, standard deviation, time under control, etc. for a given time.

In some embodiments, the recorder is operable to record information relating to movement of the device or activity of the user wearing the device. For example, the information may be raw data from the motion sensor, magnitude or direction of the movement, or estimated behavior of the user (sleeping, walking, running, etc.).

In some embodiments, the controller prohibits measurements when the device is off the body.

In some embodiments, the controller decides that the device is off the body based on sensor signal. For example, the sensor signal is one of or a combination thereof the PWV sensor(s) and the pressure sensor. No additional sensor is needed to detect that the device is off the body.

In some embodiments, the recorder is operable to record the information relating to when the device is off the body.

In some embodiments, the device is designed to be worn on a specific part of body, and, when the device is worn on other parts of the body, the controller generates an alert. The controller detects that the wearable device is worn on another part of the body based on the motion of the device detected by the motion sensor.

### I. System for Non-Invasive, Non-Occlusive Blood Pressure Monitoring Based on: Sensor Fusion, Cardiovascular Models, and Machine Learning

Some blood pressure monitoring may use (i) Oscillometric Method, (ii) Pressure-Volume Model, (iii) Oscillometric Waveform Magnitude, (iv) Characteristic Ratio Algorithm, (v) Pulse Wave Velocity, (vi) Information from Pressure Waveform, and/or (vii) Machine Learning Approach, which are described below.

### (i) Oscillometric Method

The oscillometric method relies on measurement of arterial pressure waveform (oscillations) as a response to application of varying external pressure from a cuff surrounding a segment of the arm (either upper arm or the wrist). The range of cuff pressures is from below the diastolic arterial pressure to above the systolic arterial pressure and is controlled within the feedback loop dependent on the measured oscillations. The oscillometric method has a solid theoretical base in models derived from physics and physiology.

### (ii) Pressure-Volume Model

The key component to the theory behind the oscillometric method, as well as some embodiments described herein, is the pressure-volume model. The following equation (hereinafter Eq. “(1)”) is the exponential version of the arterial pressure-volume (APV) model, which describes change of arterial volume (Vₐ₍ₜ₎) in a segment of the artery due to pressure changes: of the arterial blood vessel (Pₐ₍ₜ₎) and of the external cuff (Pₑₓₜ).

$${V}_{a} ( t ) = \{ \begin{matrix}{V}_{a .0} \cdot [ 1 + \frac{a}{b} - \frac{a}{b} \cdot exp ( - b \cdot ( {P}_{a} ( t ) - {P}_{ext} ) ) ] , & {P}_{ext} \leq {P}_{a} ( t ) \\ {V}_{a .0} \cdot exp ( - a \cdot ( {P}_{ext} - {P}_{a} ( t ) ) ) , & {P}_{ext} > {P}_{a} ( t )\end{matrix} \qquad ( 1 )$$

where a and b are model coefficients, and V_{a.0} represents reduced volume of the artery, when pressure from an external cuff equals the arterial pressure. Model coefficients a and b may be expressed as follows (hereinafter after Eq. “(2)” and Eq. “(3),” respectively):

$$\begin{matrix}a = \frac{{C}_{a . max}}{{V}_{a .0}} & ( 2 ) \\ b = \frac{{C}_{a . max}}{{V}_{a . max} - {V}_{a .0}} & ( 3 )\end{matrix}$$

where C_{a.max} is the maximum arterial compliance and V_{a.max} is the arterial volume, when the artery is fully expanded.

### (iii) Oscillometric Waveform Magnitude

The oscillometric method does not measure the arterial volume Vₐ₍ₜ₎ directly. Instead, pressure oscillations are measured using a pressure sensor. The key quantity is the Oscillometric Waveform Magnitude (OWM) defined as the difference between the maximum and minimum measured pressures. The maximum sensed pressure occurs at the time when the arterial pressure reaches the systolic level. The minimum pressure corresponds in time to the diastolic arterial pressure. The following is the relationship between the OWM and the arterial blood volume (hereinafter “Eq. 4”):

$$OWM = \frac{{V}_{a . sys} - {V}_{a . dia}}{{C}_{c}} \qquad ( 4 )$$

where V_{a.dia}, V_{a.sys} are arterial volumes corresponding to the diastolic and systolic arterial pressures, respectively. C_{c} is the cuff compliance, which may be obtained via one-time calibration as a part of product development. Eq. (1) and Eq. (4) allow one to express the OWM in terms of the model parameters. Eq. (1) describes the arterial volume with two functions depending on the relationship between the external pressure and the arterial pressure. This, in turn, leads to three different formulas for the OWM (hereinafter Eq. “(5),” Eq. “(6),” and Eq. “(7),” respectively):

$$\begin{matrix} I \text{:} & \\ OWM = \frac{{V}_{a .0}}{{C}_{c}} \cdot \frac{a}{b} \cdot [ exp ( - b \cdot {P}_{a . dia} ) - exp ( - b \cdot {P}_{a . sys} ) ] \cdot exp ( b \cdot {P}_{ext} ) & ( 5 ) \\ II \text{:} & \\ OWM = \frac{{V}_{a .0}}{{C}_{c}} \cdot [ 1 + \frac{a}{b} - \frac{a}{b} \cdot exp ( b \cdot ( {P}_{ext} - {P}_{a . sys} ) ) - exp ( a \cdot ( {P}_{a . dia} - {P}_{ext} ) ) ] & ( 6 ) \\ I II \text{:} & \\ OWM = \frac{{V}_{a .0}}{{C}_{c}} \cdot [ exp ( a \cdot {P}_{a . sys} ) - exp ( a \cdot {P}_{a . dia} ) ] \cdot exp ( - a \cdot {P}_{ext} ) & ( 7 )\end{matrix}$$

with the following definition of the three pressure ranges (hereinafter Eq. “(8),” Eq. “(9),” and Eq. “(10),” respectively):

I:Pₑₓₜ≤P_{a.dia} (8)

II:P_{a.dia}<Pₑₓₜ<P_{a.sys} (9)

III:P_{a.sys}≤Pₑₓₜ (10)

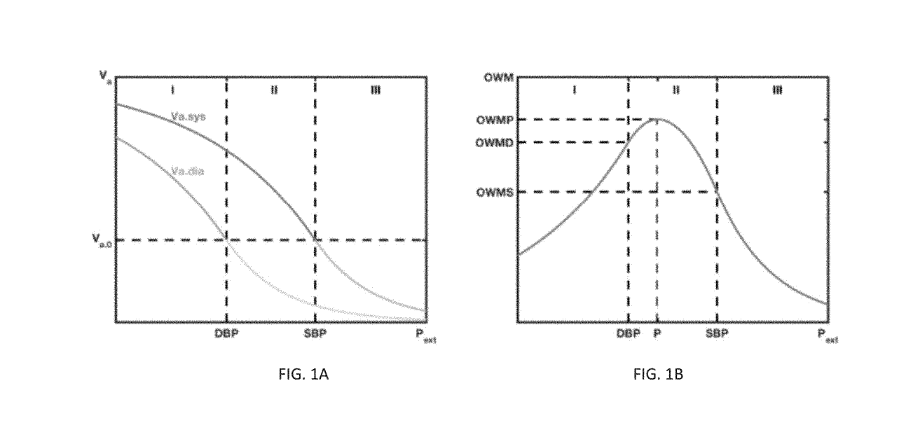

Sample arterial volume and the corresponding OWM as functions of the external pressure are shown in FIGS. 1A-B.

### (iv) Characteristic Ratio Algorithm

The characteristic ratio algorithm assumes a particular relationship between the peak OWM (OWMP) and the OWM at external pressures that are equal to the diastolic (OWMD) and systolic (OWMS) arterial pressures. These relationships may be written as follows (hereinafter Eq. “(11)” and Eq. “(12)”, respectively):

OWMD=κ_{dia}·OWMP (11)

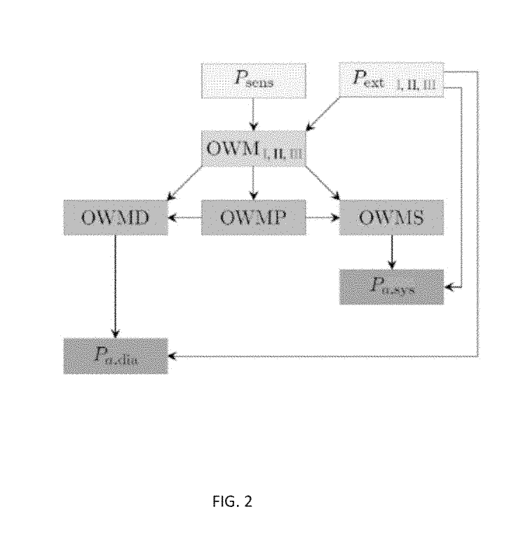

OWMS=κ_{sys}·OWMP (12)

where κ_{dia} and κ_{sys} are constants determined empirically. Typical values for coefficients κ_{dia}, κ_{sys} are 0.82, 0.55, respectively. For improved accuracy, some researchers suggest to make κ_{dia}, κ_{sys} functions of MAP. Both methods are used in practical blood pressure monitors giving accuracy within the FDA requirements. The external pressure is swept mostly within range II but slightly entering ranges I and III. In this process, the peak OWM (OWMP) is determined. A lower external pressure (lower than point P in FIG. 1B) corresponding to OWMD calculated with Eq. (11) is interpreted as the diastolic blood pressure. A higher external pressure corresponding to OWMS calculated with Eq. (12) is interpreted as the systolic blood pressure. FIG. 2 shows the calculation flow for the oscillometric method.

### (v) Pulse Wave Velocity

Propagation of pressure waves in the cardiovascular system is described by the cardiovascular fluid dynamics. It was observed that, under certain conditions, pulse wave velocity (PWV) is correlated with the blood pressure (either diastolic, systolic, or MAP). This observation led to many efforts to build a cuff-less system inferring blood pressure strictly from the PWV. The PWV may be measured using various technologies, either directly or indirectly from the time delay between pulses measured at different locations and the distance between these locations. Technologies related to the direct measurements include various types of radars (RF or laser). Indirect measurements may involve two photo-plethysmography (PPG) sensors, or electrocardiography (ECG) and the PPG, or methods with the PPG sensor replaced by a pressure sensor. Attractiveness of such techniques is that they are cuff-less and allow continuous blood pressure monitoring. However, one common conclusion from these studies is that the method based on measurement of the PWV requires frequent calibration (typically done with the oscillometric method). Typically, with this method alone, it is hard to achieve FDA required accuracy even with calibration. Accuracy may be improved, and calibrations may be less frequent, in case the system is used on patients in a very steady state. For instance, the application may be limited to ICU patients laying on a hospital bed.

PWV may be expressed using the same terms, as in the oscillometry method (hereinafter Eq. “(13)”):

$$PWV = \sqrt{\frac{{V}_{a}}{{ρ}_{b}} \cdot \frac{\partial {P}_{a}}{\partial {V}_{a}}} \qquad ( 13 )$$

where ρ_{b=1025} kg is the blood density. From (1) and (13) we get (hereinafter Eq. “(14)”):

$$PWV = \sqrt{\frac{1}{{ρ}_{b}} \cdot ( ( \frac{1}{a} + \frac{1}{b} ) \cdot exp ( b \cdot ( {P}_{a} - {P}_{ext} ) ) - \frac{1}{b} )} \qquad ( 14 )$$

Eq. (14) shows that the PWV is indeed related with Pₐ. As Pₐ is a function of time, PWV must also change in time. This means that the pulse wave is subject to dispersion. In other words, wave points corresponding to the higher arterial pressures (e.g., P_{asys}) are traveling at higher speeds than the wave points corresponding to the lower arterial pressures (e.g., P_{a.dia}). Typically, one relates the PWV to P_{a.dia} by looking at the propagation of the dip point in the OWM. This method has also an advantage of being less influenced by reflected waves.

It is important to note that the PWV in Eq. (14) depends not only on Pₐ but also on variables a and b. It should be stressed that a and b are variables and not constants. It can be clearly seen from eqs. (2) and (3), which define these coefficients. Out of three parameters in these equations (C_{a.max}, V_{a.max}, V_{a.0}), only V_{a.0} may be considered constant per patient. The other parameters (C_{a.max} and V_{a.max}) may be constant only when the patient is in a steady state. This is the reason why the method involving measurement of the PWV may work with the calibration but fails when calibration is not in the picture.

In some embodiments, the PWV method is used as a part of a larger system. Only in combination with other sources of information, this leads to accurate inference of blood pressure.

### (vi) Information from Pressure Waveforms

Researchers also try to augment the method based on measurement of the PWV by information found in the pressure waveforms. Different kinds of pressure waveforms may be measured at different locations. This includes arterial pressure waveforms typically measured with pressure sensors, and capillarian pressure waveforms measured with the PPG. In general, identification of cardiovascular parameters from the pressure waveform belongs to a class of so-called inverse problems. Such problems are hard to solve, unless using machine-learning techniques, which in this case require a massive amount of high quality data. To date, no calibration-less method involving the PWV and the pressure waveforms has successfully met the accuracy requirements for blood pressure.

### (vii) Machine Learning Approach

In practice, features from the pressure waveforms are included in the model using machine-learning techniques. Machine learning builds models from data. When there is sufficient amount of data, models may be built even without any prior knowledge about the system. Typically, features used in the model are carefully engineered and are extracted from measured signals. More advanced machine learning techniques (mostly from the category of deep learning) have capability to not only derive models for inference of blood pressure from engineered features but also identify the relevant features directly from either raw or slightly pre-processed data. Such techniques require much larger datasets for sufficient training of the models.

A large drawback of those machine-learning techniques is that their final accuracy cannot be estimated up-front without availability of data. In practice, this means that the development process may be very expensive, time consuming, and risky. In order to find out whether a particular set of sensors is sufficient for accurate inference of blood pressure, one has to first build high quality prototypes, collect a massive amount of clinical data (thousands and tens of thousands) on a representative population of subjects, and then apply the machine learning techniques. It is not known up-front whether this process will be successful in terms of meeting the FDA accuracy requirements. To date, no calibration-less and cuff-less method involving the machine learning techniques has successfully met the accuracy requirements for blood pressure.

In some embodiments, machine learning techniques are used on top of a system of physiological and physical models that already have sufficient information for blood pressure, and can achieve FDA required accuracy based on a relatively small amount of data (a few hundred); it can further improve accuracy as the database increases.

Methods and devices for continuous non-invasive, non-occlusive blood pressure monitoring using one or a plurality of sensor fusion, cardiovascular modeling, and machine learning techniques. Accurate blood pressure can be obtained through measuring sensor data with regards to low pressure actuation (below the said diastolic pressure of the said user) and processing the data based on the contexts of one or a plurality of features such as pulse wave velocity, oscillometric waveforms, pressure-volume model, empirical ratios, biometrics and other characterizing features. One feature of some embodiments is to apply physiological equations or simulation models to utilize the said features to solve blood pressure mathematically or numerically. Another embodiment is to apply machine learning on the said features, such as self-learning, feature engineering, deep-learning, to calculate blood pressure. Yet another embodiment is to combine physiological equations or simulation models with machine learning, to calculate blood pressure. Yet another embodiment is a sensor fusion device that incorporates at least low-pressure actuation, ECG and PPG sensors to generate and record signals that enable non-invasive, non-occlusive blood pressure monitoring. Yet another embodiment is a device that utilizes one or a plurality of the above embodiments in combination with pulse wave velocity, oscillometric waveforms, and other methods for frequent blood pressure monitoring.

Some embodiments described herein feature a system for inferencing blood pressure based on oscillometric waveform signals without the need to measure at varying external pressures all the way from below diastolic pressure to above systolic pressure or vice versa as oscillometry. By combining measurements of the PWV, the said system uses one segment of oscillometric waveforms to extrapolate physiologically sufficient information to infer all the necessary parameters of oscillometry, which can be further improved through feature extraction and machine learning, and thus achieve highly accurate inference of blood pressure. The one segment of chosen oscillometric waveforms can be any segment below the diastolic pressure, between the diastolic pressure and systolic pressure, above the systolic pressure, or a combination of the above that does not require varying the eternal pressure from below diastolic pressure to above systolic pressure or vice versa. The advantage of this system is enabling the use of miniaturized pressure actuation systems that only work within a small range of pressure change, such as 30 mmHg, rather than 300 mmHg. Moreover, the system demonstrates tremendous robustness and consistency in extrapolating the full physiological information of inferencing blood pressure regardless of the external pressure to take the segment of waveforms, which means blood pressure can be inferred at very low external pressure for very comfortable, non-occlusive, 24-hour monitoring.

An objective of some embodiments is to provide a system of software and hardware for comfortable, non-invasive, non-occlusive blood pressure monitoring based on one or a plurality of sensor fusion, cardiovascular modeling, and machine learning techniques that exploits full physiological information for inference of blood pressure, while significantly reducing the range of external pressure change needed as compared to the oscillometric method. The oscillometric method requires exerting pressures exceeding the systolic pressure of said user to occlude artery, which requires a working range of 0 to as high as 300 mmHg of external pressure for covering the needs of use cases. Some embodiments only uses one segment of the oscillometric waveforms, and optimizes the range of external pressure change to a segment in pressure region I (see Eq. (5) and FIGS. 1A-B) for non-occlusive and comfortable measurement. Further, without the need for a working range of 0 to 300 mmHg, the pressure actuation system can be optimized for low power consumption and miniaturized design and enable comfortable, accurate, and wearable blood pressure monitoring devices.

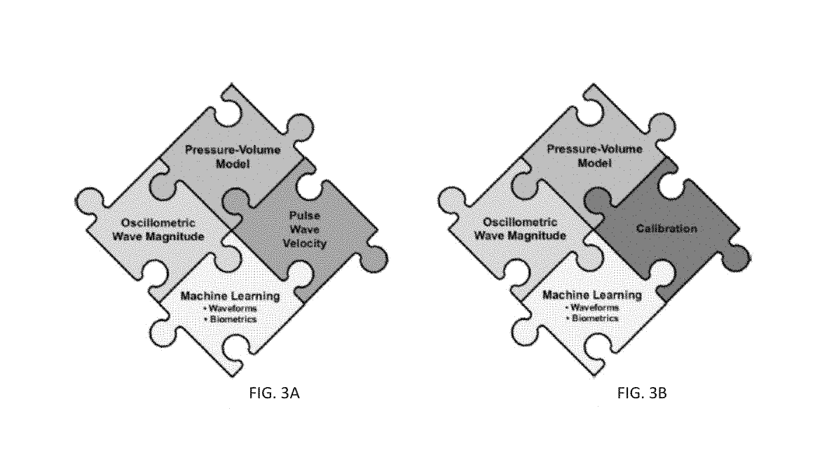

In order to preserve the potential for high accuracy, some embodiments rely on the same physics and physiology-based framework of equations as the oscillometric method. However, some embodiments differ from some approaches in that it only uses part of the waveform signals in response to non-occlusive external pressure and can optimize on its own and reduce the range of working pressure significantly. Thus, hardware devices that contain pressure actuator of smaller power or in miniaturized form can be used, and external pressures can be applied through contact with a reduced skin area close to the artery. In addition, to ensure sufficient information, the low-pressure oscillometry is augmented by other sources of information—in particular: PWV method, or one-time-per-patient calibration—in order to achieve adequate accuracy. The accuracy is further improved using machine learning techniques applied to the waveforms captured by the sensors and to biometric information provided by the user. FIGS. 3A-B show the key elements of the algorithm for the case involving the PWV method and the case with one-time-per-patient calibration.

Low Pressure Oscillometry. One should note that the OWM corresponding to the pressure regions I (Eq. (5)) and III (Eq. (7)) are pure exponential functions of the external pressure. Therefore, by applying log function to both sides of these equations, we get a linear system (hereinafter Eq. “(15)” and Eq. “(16),” respectively):

I:log(OWM)=b·Pₑₓₜ+c (15)

III:log(OWM)=−a·Pₑₓₜ+d (16)

where coefficients c and d are defined as follows (hereinafter Eq. “(17)” and Eq. “(18)”, respectively):

$$\begin{matrix}exp ( c ) = \frac{{V}_{a .0}}{{C}_{c}} \cdot \frac{a}{b} \cdot [ exp ( - b \cdot {P}_{a . dia} ) - exp ( - b \cdot {P}_{a . sys} ) ] & ( 17 ) \\ exp ( d ) = \frac{{V}_{a .0}}{{C}_{c}} \cdot [ exp ( a \cdot {P}_{a . sys} ) - exp ( a \cdot {P}_{a . dia} ) ] & ( 18 )\end{matrix}$$

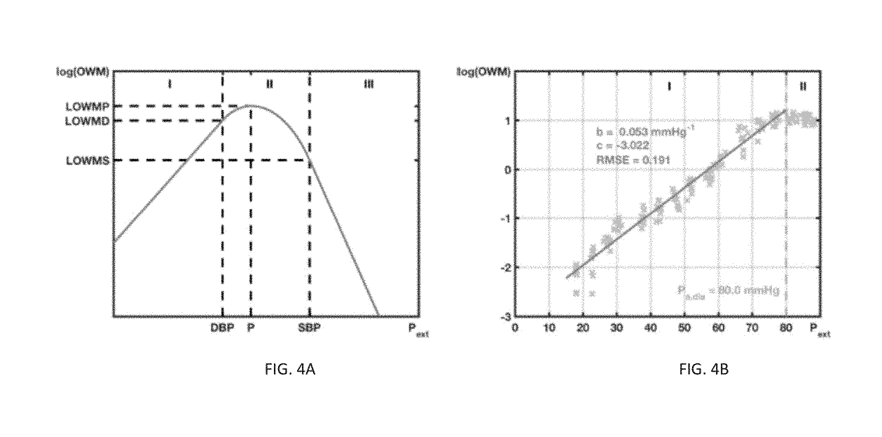

Linearity of log (OWM) as a function of Pₑₓₜ can be clearly seen in pressure ranges I and III in FIG. 4. Eqs. (15), (16) may be solved given at least two values of Pₑₓₜ (and the corresponding OWMs) per pressure range (I and III). If the number of external pressures is larger than two, linear regression should be used to find optimal function that fits with parameters b, c, a, and d. In some embodiments, the system involves low-pressure oscillometry, i.e., it limits external pressures to values below the diastolic pressure (region I). OWM levels are measured for low external pressures and linear regression is applied to Eq. (15). This yields coefficients b and c. Inverse characteristic ratio. Values of OWMD, OWMP, and OWMS in eqs. (11), (12) may be calculated from (5), (6), and (7). This gives the following relationship (hereinafter Eq. “(19)”):

$$\begin{matrix}{κ}_{dia}^{- 1} \cdot \frac{a}{b} \cdot [ 1 - exp ( - b \cdot {P}_{a . pulse} ) ] = \\ ( 1 + \frac{a}{b} ) \cdot [ 1 - exp ( - \frac{a \cdot b}{a + b} \cdot {P}_{a . pulse} ) ] = {κ}_{sys}^{- 1} \cdot [ 1 - exp ( - a \cdot {P}_{a . pulse} ) ] \end{matrix} \qquad ( 19 )$$

where P_{a.pulse} is the pulse pressure (hereinafter Eq. “(20)”):

P_{a.pulse}=P_{a.sys}−P_{a.dia} (20)

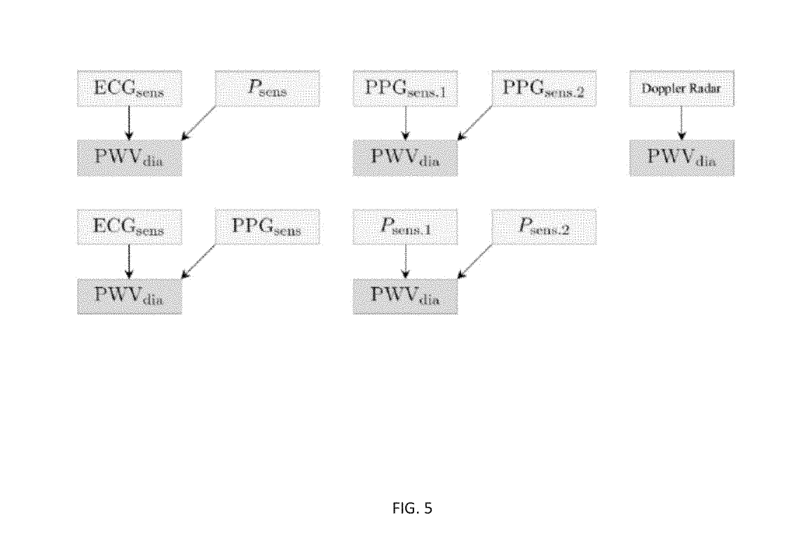

Given parameter b (identified earlier from the linear regression applied to Eq. (15)), formula (19) may be uniquely solved for a and P_{a.pulse}. The solution can be based on numerical techniques. The results can be tabularized with respect to b, and therefore calculations do not have to be repeated. The system can use a lookup table with b as an argument and a, P_{a.pulse} as the output. Pulse Wave Velocity. The PWV can be related to the diastolic pressure (further denoted by PWV_{dia}) by looking at the propagation of the dip point in the pressure waveforms. FIG. 5 shows different ways to determine PWV_{dia} from different combinations of sensors: ECG and pressure sensor, ECG and PPG, two spatially separated pressure or PPG sensors, Doppler radar. With known values of b, a, P_{a.pulse}, and PWV_{dia}, one can calculate P_{a.dia} by rearranging terms in Eq. (14) to get (hereinafter Eq. “(21)”):

$${P}_{a . dia} = {P}_{ext} + \frac{1}{b} \cdot ln ( \frac{a \cdot b \cdot {ρ}_{b} \cdot {PWV}_{dia}^{2} + a}{a + b} ) \qquad ( 21 )$$

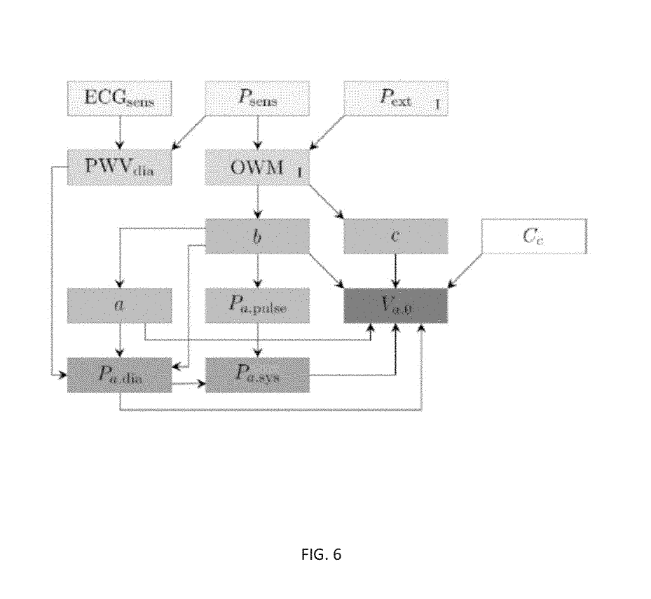

with all the quantities on the right hand side already identified as described earlier. Completeness of Information. The procedure described above gives values to quantities b, a, P_{a.pulse}, and P_{a.dia}. From Eq. (20), one can also calculate P_{a.sys} as follows (hereinafter Eq. “(22)”):
With diastolic and systolic pressure defined, this gives complete information required from a blood pressure monitor. Moreover, parameters a and b also carry physiological information describing the vascular tone. This information may be displayed to the patient and/or to the doctor to provide additional insight on the patient's vascular system. FIG. 6 shows the full calculation flow described above. Note that a particular way of inference of PWV_{dia} was assumed (from the ECG and the pressure waveform). Other methods for inference of PWV_{dia} can be used as shown earlier in FIG. 5.

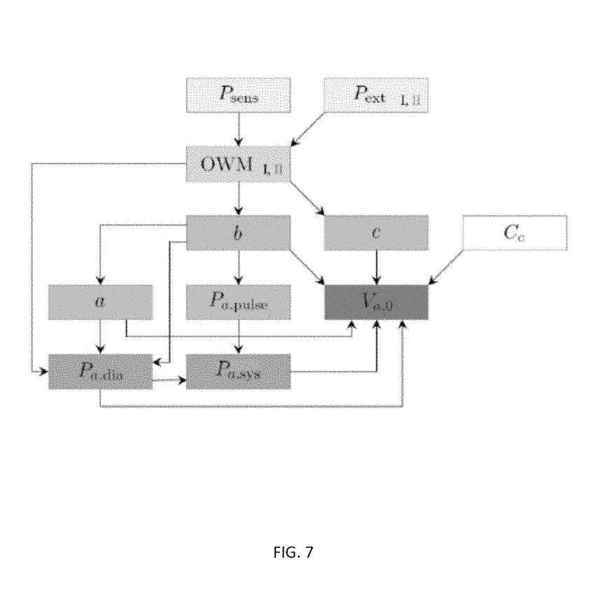

Extended algorithms. The inference of blood pressure with a segment from region II or regions II and III can be processed the same way as in region I because the symmetrical nature of the equations. Taking measurements at those regions requires higher external pressure to be applied to the artery, which do not have the advantage as region I in real application. However, as an alternative to the PWV based calculations, P_{a.dia} may be obtained by extending the low-pressure oscillometry into pressure region II. As shown earlier in FIG. 4, the linearity of log (OWM) versus the external pressure in region I breaks when the external pressure enters region II, or at the point where the external pressure equals the diastolic blood pressure. This trend change may be easily identified, and therefore the diastolic pressure may be determined. FIG. 7 shows the calculation flow involving extended low-pressure oscillometry. On the other hand, the linearity of log (OWM) in region I also enables the identification of an effective pressure range that starts from the lowest pressure exhibiting such linearity to the lowest pressure where the linearity becomes steady. Through signal processing, the system can optimize on its own and identify the maximum external pressure required by the system to achieve the FDA accuracy, which can be reduced to much lower than the diastolic pressure of the said user. The system can incorporate the signal processing algorithm to make the pressure exertion dynamic and intelligent to achieve the lowest necessary pressure and maximum comfort.

Self-learning. Furthermore, Eq. (17) and coefficient c determined via linear regression (15) may be used to calculate the reduced blood volume V_{a.0} (hereinafter Eq. “(23)”):

$${V}_{a .0} = \frac{{C}_{c} \cdot \frac{b}{a} \cdot exp ( c )}{exp ( - b \cdot {P}_{a . dia} ) - exp ( - b \cdot {P}_{a . sys} )} \qquad ( 23 )$$

With quantities b, a, and V^{a.0}, one can also calculate parameters V_{a.max} and C_{a.max}, which may be displayed to the patient and/or to the doctor. From (2) and (3), we get (hereinafter Eq. “(24)” and Eq. “(25),” respectively):

$$\begin{matrix}{C}_{a . max} = a \cdot {V}_{a .0} & ( 24 ) \\ {V}_{a . max} = ( 1 + \frac{a}{b} ) \cdot {V}_{a .0} & ( 25 )\end{matrix}$$

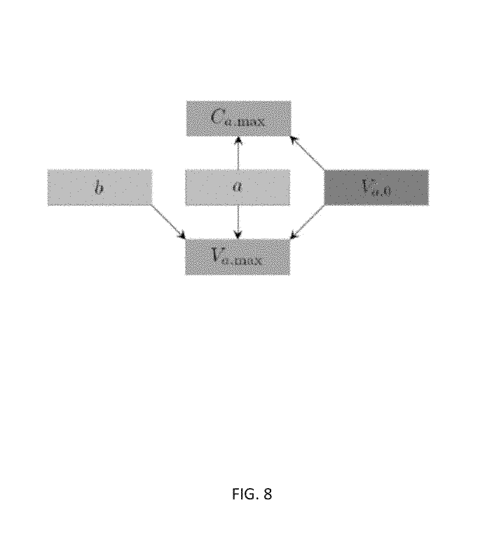

This calculation flow is also shown in FIG. 8.

It should be noted that parameter V_{a.0} is an anatomical constant. In other words, this quantity does not vary from one measurement to another as long as the measurements are done on the same patient. This fact can be used to improve system accuracy over time. For instance, values of V_{a.0} from (23) may be averaged over a number of initial measurements. Averaging decreases individual errors and, therefore, after several measurements, V_{a.0} may be considered known with a high level of accuracy. At that point, Eq. (23) may be used as an alternative way to identify one of the other variables (a, b, P_{a.pulse}, P_{a.dia}, P_{a.sys}). As this leads to multiple ways of getting the same final quantity, a weighted average can be used to get one equation with accuracy better than that from the individual terms. The weights in the averaging may be established via machine learning (linear regression) to give the best balance and the lowest error. As an example, one may consider calculation of P_{a.dia} using (15) to get (hereinafter Eq. “(26)”):

$${P}_{a . dia} = \frac{1}{b} \cdot ln [ \frac{{V}_{a .0}}{{C}_{c}} \cdot \frac{a}{b} \cdot exp ( c ) \cdot [ 1 - exp ( - b \cdot {P}_{a . pulse} ) ] ] \qquad ( 26 )$$

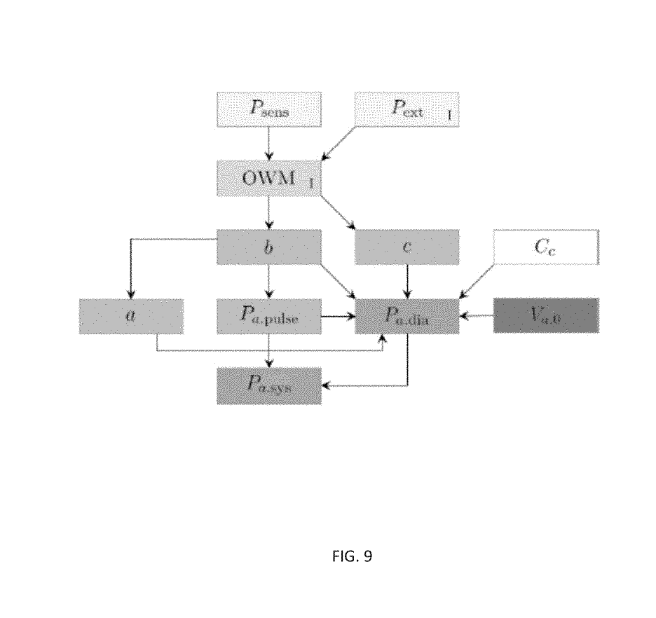

Eq. (26) is an alternative to (21). This enables more accurate determination of P_{a.dia} as a weighted average of results from (21) and (26). Alternatively, Eq. (26) may be considered a replacement for (21), which makes determination of PWV optional after initial measurements. This alternative mode of operation is especially attractive, when inference of PWV involves sensing the ECG signal. Measurement of ECG using a compact system typically requires user involvement (e.g., placement of the index finger on one of the ECG electrodes). By making this signal optional, measurement becomes less dependent on the user. Full calculation flow without involvement of the PWV is shown in FIG. 9.

System involving calibration. By taking yet another view on Eq. (26), it may be treated as a replacement for Eq. (21) in all measurements. In such a case, one needs to know the value of anatomical constant V_{a.0} up-front.

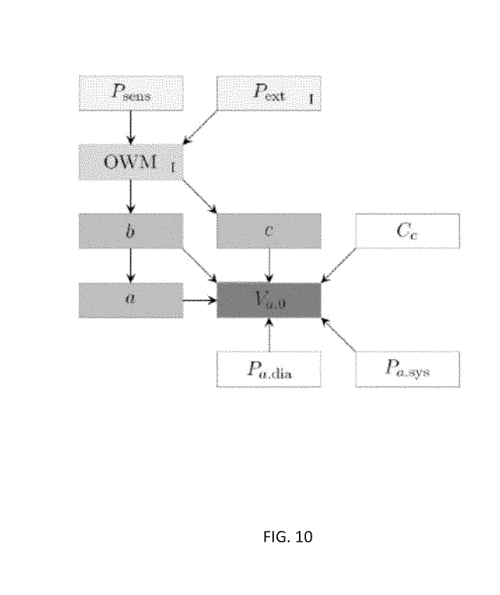

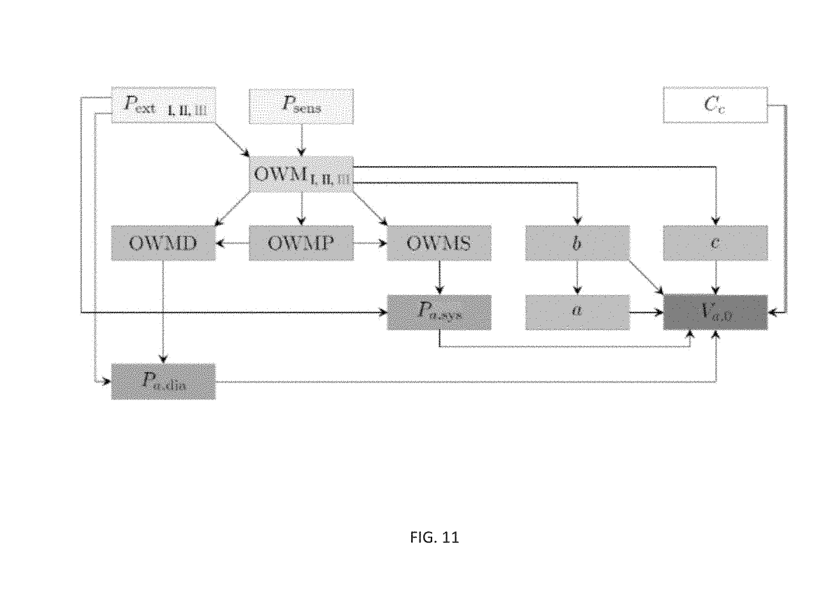

This can be done via a one-time-per-user calibration. Number of calibration methods are possible. In general, a scan involving the system to be calibrated should be paired with accurate (reference) determination of blood pressure by other means (e.g., the oscillometric or auscultatoric method). V_{a.0} can be then determined from Eq. (23) using quantities b, a obtained with the low-pressure oscillometry, and P_{a.dia}, P_{a.sys} from the reference method. FIG. 10 shows calculation flow during the one-time-per-user calibration using an external reference system. However, by temporarily extending pressure range of the external actuator, one can get the reference values of P_{a.dia} and P_{a.sys} using the same system as normally used for the low-pressure oscillometry. By extending the pressure range to levels above diastolic pressure (i.e., by entering region II), one can use calculation flow shown earlier in FIG. 7. If the pressure range is further extended above the systolic level, oscillometry may be involved and one can use calculation flow shown in FIG. 11.

Machine Learning. Accuracy of the system may be further improved using machine learning techniques applied to all signals captured by the system as well as biometric information (such as age, weight, height, sex) entered by the user. This should be implemented at the top of the low-pressure oscillometry and measurement of PWV, which assures that the required accuracy will be met.

First, all parameters from equations above (equations (15), (17), (19)-(26)) serve as features for machine learning. Secondly, machine learning can identify a mixture of features varying from one scan to another and features, which are constant for a given subject. Use of the later features enables learning from earlier measurements. The constant features may be identified with accuracy increasing with the number of measurements performed on the subject. Inference of blood pressure from both sets of features can be established via supervised machine learning (e.g., supervised deep learning).

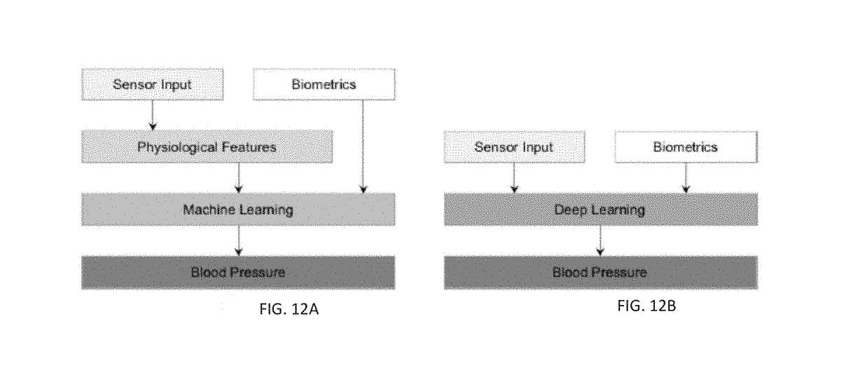

With large enough data-set, machine learning can build the entire model. This means that the computation flow based on physical/physiological equations may be replaced by one model learned directly from data. The fact that the theoretical model exists guarantees feasibility with this approach. FIG. 12 shows a general computation flow for the machine learning approach.

Hybrid Approach. Previous examples described herein included running the blood pressure inference in different modes over different scans. In particular, self-learning and use of calibration mode were introduced. The hybrid approach to the measurements may be expanded further. As noted earlier, when a patient is in a steady state, coefficients a, b are relatively constant. Per Eq. (19), P_{a.pulse} is also relatively constant. This means that one can infer P_{a.dia} using only measurement of PWV_{dia} and Eq. (21), and then calculate P_{a.sys} from Eq. (22). The system will use low-pressure oscillometry whenever it believes that the patient's state differs from the state corresponding to the previous measurement. Otherwise, a simpler scan involving only measurement of PWV_{dia} and use of previous values of a and b may be performed. Such simpler measurement can also be used for beat-to-beat estimates of blood pressure in between the measurements involving the low-pressure oscillometry.

Assessment of patient's state changes may be based on data from accelerometer and/or on the pulse rate history (determined from the PPG signal). Relatively steady state may be assumed during sleep.

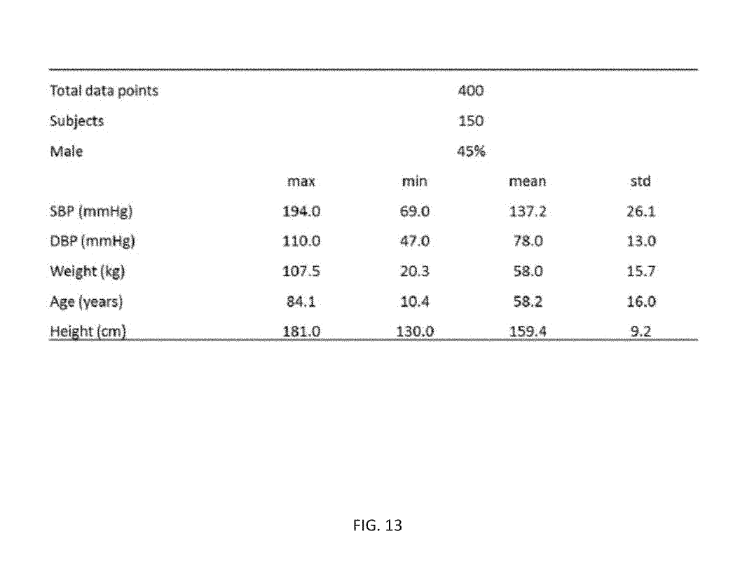

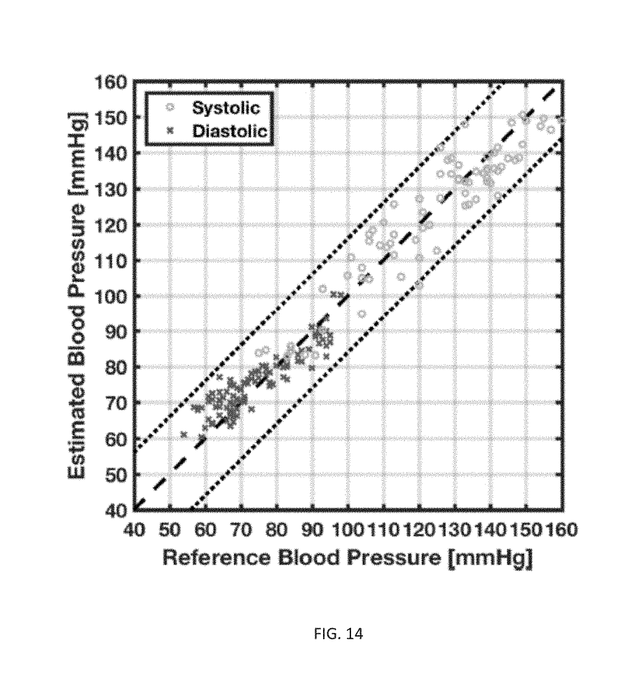

Data Validation. Validation of the system was performed on a prototype built with an Arduino Mega board, a small diaphragm pump, a small air valve, a blood pressure monitoring cuff that wraps around the left arms of test subjects, two PPG sensors that put on the middle finger and under the cuff respectively, and a one-lead ECG sensor that are attached to the subjects' two arms and left leg. Omron BP 761 was used to measure reference blood pressure. There were 400 measurements collected from 150 subjects, and the statistics of those subjects are in FIG. 13. The data were separated randomly into training and validation sets before analysis. In the validation set of 40 subjects, the calculated diastolic pressure has a mean square error of 4.5 mmHg and the systolic pressure 8.1 mmHg, when compared with reference values. FIG. 14 shows plots of the estimated blood pressure versus the reference blood pressure. The two dotted lines mark 95% confidence interval of the standard requirements recognized by the FDA. The mean standard error for systolic pressure in the validation data is 8.1 mmHg, while the mean standard error for diastolic pressure is 4.5 mmHg. Most of the data is within the 95% confidence interval of the FDA requirements denoted by the dotted lines. With a larger number of subjects and more data points, the self-learning mechanism of the system can be triggered to improve the accuracy further.

### Hardware

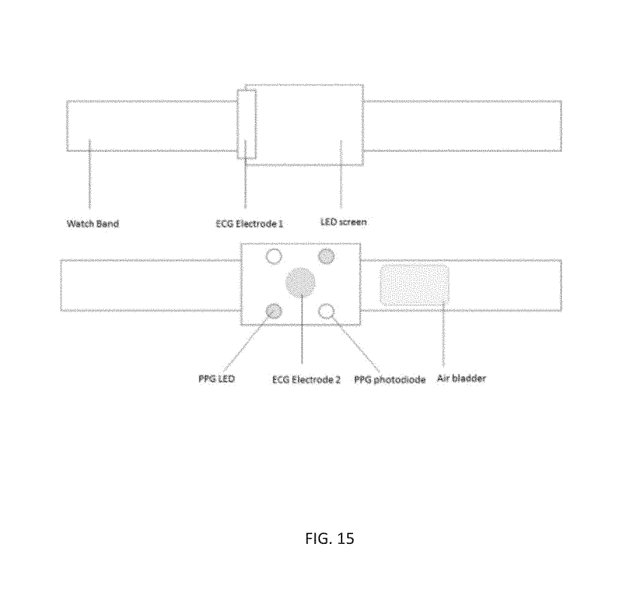

Sensor Fusion. FIG. 15 shows one embodiment of the comfortable, non-occlusive blood pressure monitoring system: a wrist worn 24/7 blood pressure device. This system is designed for wearing at the left wrist. The system can be also designed to be worn at the right wrist. The PPG sensors are placed under the body of the electronic component.

The wristband includes: low-pressure cellular actuator possibly linked to multiple independent pressure sensors, two PPG sensors intended to touch the skin at the bottom of the wrist, and an optional 2-electrode ECG sensor (one electrode at the inner and one on the outer side of the band). This system applies a low pressure to the wrist in the region of the artery. The pressure is below the diastolic level. During the external pressure application, the system measures the response of the artery using the pressure sensor. This is similar technique to oscillometry, except at a much lower pressure range (the “low-pressure oscillometry”). Simultaneously with sensing pressure, the system collects the ECG and the PPG signals. They are used in two ways for two estimates of the PWV. Measurements involving the ECG are optional. In one embodiment, the low pressure actuation is realized by connecting a miniaturized pump such as piezoelectric pump (for example, 21 mm×19 mm×4.5 mm) and a miniaturized valve (for example, 16 mm×19 mm×4 mm) and controlling the pumping and releasing of air.

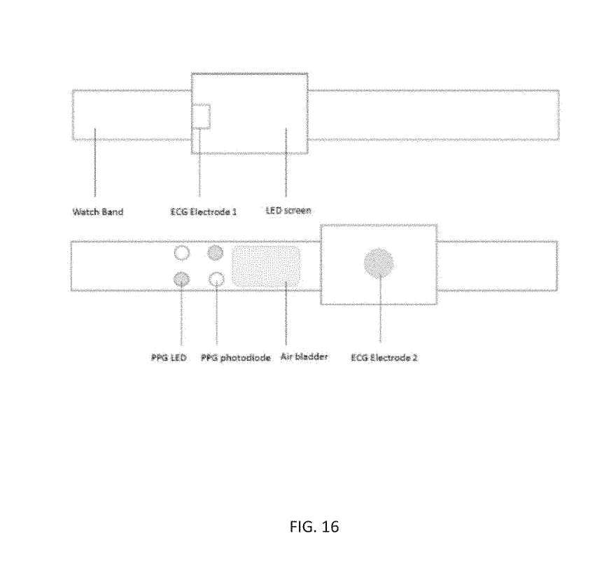

FIG. 16 is another embodiment of the system in a 24/7 wristband (worn by the left hand). This system is designed for wearing at the left wrist. The system can be also designed to be worn at the right wrist. The PPG sensors are placed under the band.

The wristband includes: low-pressure cellular actuator possibly linked to multiple independent pressure sensors, optional 2-electrode ECG sensor (one electrode at the inner and one on the outer side of the band), and two PPG sensors that are attached to the band so that they touch the skin at the inside of the wrist when the electronic component is worn on the back of the wrist like a watch. The low pressure actuation is realized by connecting a miniaturized pump such as piezoelectric pump (for example, 21 mm×19 mm×4.5 mm) and a miniaturized valve (for example, 16 mm×19 mm×4 mm) and controlling the pumping and releasing of air.

Another embodiment of the system is to incorporate the methods into stationary blood pressure monitors, 24-hour ambulance monitors or patient monitors, collecting required signals through those devices with optional addition of PPG or ECG sensors. The cuff and pneumatic system of the known blood pressure monitors can be served as the pressure actuator for applying low external pressure. Optional PPG sensors can be placed under the cuff, on the fingers, on the ear loops or anywhere that yields good quality PPG signal. Optional ECG signals can be collected from the ECG sensors from a patient monitor, a Holter monitor, a wearable ECG pad or ECG sensing fabrics. The signal processing and machine learning methods enable a non-occlusive, comfortable monitoring of 24-hour blood pressure to improve patient compliance and acceptability of the use of those devices. The advantage of this system is that the algorithm can be incorporated into existing devices with minimal changes of hardware and development time, and hospitals and clinics can quickly adopt the system with very low upfront upgrading costs, while providing much better comfort and ease for patients.

Low-Pressure Oscillometry. The following is a list of hardware to support low-pressure oscillometry, according to various embodiments:
A wrist-worn device with a pneumatic system and electronics compartment.
The pneumatic system consists of an air bladder that applies changing unloading pressure on top of the artery, a pressure application and releasing mechanism, and a pressure sensor.
The pressure application and releasing mechanism may be a micro pump, piezoelectric pump, servomotor, or memory shape alloy.
The pressure sensor could be replaced by a strain gauge.
The unloading pressure can be applied through an air bladder that wraps around the wrist.
The unloading pressure can be applied by an air bladder as small as only covering the skin on top of the radial artery.
The unloading pressure can be applied by a larger air bladder that covers part of the full periphery of the wrist.
The unloading pressure can be applied by one or more small air pockets that are placed on specific areas of the wrist, such as on top of one or more arteries, or one or more veins, or a combination of arteries and veins.
The unloading pressure can be either an increasing or decreasing pressure.
The unloading pressure can be discrete pressures in the form of two or more pressure steps, or continuously changing pressure.
The main embodiment of the unloading pressure is below the diastolic pressure, but it can be in between the diastolic and systolic.
The pressure application and releasing mechanism may be calibrated so that the air volume in and out of the pneumatic system can be calculated to provide additional input to the algorithm.
Additional sensors, such as radar, ultra wide band sensors, or ultrasound sensors can be used to characterize the distension and diameter of the artery, to calibrate for the variability for different measurements and individuals.
Calibration can be also obtained through a separate blood pressure monitor device, invasive arterial line blood pressure, or Korotkoff sounds.
Calibration can be done through the device itself by raising the pressure above systolic and release to below diastolic to obtain the full oscillometric waveform.
Calibration can be done through the device by raising the pressure to in between systolic and diastolic.
Calibration can be done through an add-on device or module that can raise pressures to above diastolic pressure.

Pulse Wave Velocity. The following is a list of hardware to support measurement of the PWV, according to various embodiments:
PWV can be obtained through the combination of an ECG with a PPG incorporated into the electronic part of the device.
PWV can be obtained through the combination of an ECG with pressure waves obtained by the pressure sensor connected to the air bladder.
PWV can be obtained through two PPGs that are placed at two different locations along the artery.
PWV can be obtained through two pressure sensor and two air bladders that are placed at two different locations along the artery.
PWV can be obtained using a Doppler radar (laser or RF).
Two or more PPG sensors can be placed around the wrist to sense the different strength of signals, varied with location, thickness of skin and tissue, etc.
A combination of the two kinds of PPG placements can be implemented in the system to form an array of PPG sensors.
The light source in the PPG sensor can use one or a mix of wavelengths, such as IR, red, blue, green, wideband, etc., which have different penetration depths to detect pressure waveforms from blood vessels at various depths.
The radiation pattern of the light source and the photodetector may have the main lobe pointing perpendicularly or at an angle with respect to the skin surface.
Two electrode ECG, one on top of the wristband, and one underneath attaching the skin, is used to capture the ECG signal.
ECG of three or more electrodes can be connected to the wristband to capture ECG signal.
ECG of two or three electrodes can be placed around the upper arm to obtain single arm ECG signals.
The placement of the sensors can be anywhere in the body that a useful signal is captured. PPGs can be placed on fingertips, earlobes, wrists, or arms. ECG electrodes can be placed on chests, legs, arms, shoulders, or forehead.

Other components. The following is a list of the other system components, according to various embodiments:
The electronics compartment contains a microprocessor, a battery, an optional LED screen, BLE or Wi-Fi module for mobile connectivity, and a memory for storing data.
The device will be able to connect to the cloud and share data.
Piezoelectric sensors or strain gauge can be used to sense pulse pressures in the system.
Accelerator and gyroscope can be used to detect the motion of arms and help filter motion artifacts.
Accelerator and gyroscope can be used to perform compensation for pressure changes associated with the altitude and gravity.

### Examples

Example 1 is a blood pressure monitoring system, utilizing one segment of oscillometric waveforms, in addition to one or a plurality of parameters or features from pulse wave velocity, artery pressure-volume relationships, empirical ratios, and user biometrics to inference accurate blood pressure, and enable non-invasive, and non-occlusive 24/7 monitoring. Thanks to the robustness of using low pressure segment of oscillometric waveforms to achieve the full physiological information as obtained by a full oscillometric waveform that spans from below the diastolic pressure to above the systolic pressure, the system can be operated in much smaller range and lower external pressures and enable miniaturized hardware devices in the form of wristbands.

Example 2 includes the subject matter of example 1 (or any other example herein), the system further to collect and process sensor data and provide diastolic and systolic readings. One embodiment is a wristband with miniaturized pressure actuation system and optional placement of PPG and ECG signals. Another embodiment is stationary blood pressure monitors and patient monitors that exert low pressure actuation to achieve comfortable blood pressure monitoring.

Example 3 includes the subject matter of any of examples 1-2 (or any other example herein), the system further to collect and process sensor data and provide pressure assessment, such as low, medium, and high.

Example 4 includes the subject matter of any of examples 1-3 (or any other example herein), where physiological equations or simulation models are utilized to solve blood pressure mathematically or numerically.

Example 5 includes the subject matter of any of examples 1-4 (or any other example herein), where machine learning, such as self-learning, feature engineering, deep-learning, is utilized to calculate blood pressure.

Example 6 includes the subject matter of any of examples 1-5 (or any other example herein), where a combination of physiological equations or simulation models with machine learning are utilized to calculate blood pressure.

Example 7 includes the subject matter of any of example 1-6 (or any other example herein), where calibration is performed to provide inputs to the blood pressure calculation.

Example 8 includes the subject matter of any of examples 1-7 (or any other example herein), where a sensor fusion device that incorporates at least low-pressure actuation, ECG and PPG sensors to generate and record sensor signals.

Example 9 includes the subject matter of any of examples 1-8 (or any other example herein), where a device can use other methods, such as pulse wave velocity or oscillometric waveforms for blood pressure measurements, in between the measurements by the said methods.

### II. Low Pressure Actuation Blood Pressure Monitoring

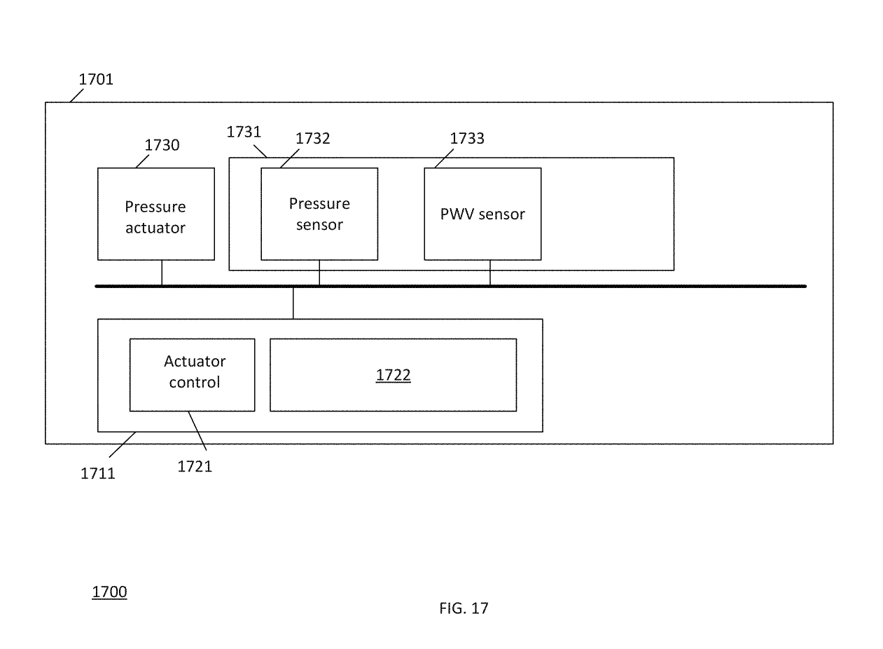

FIG. 17 is a block diagram of a low pressure range blood monitoring system 1700 including a wearable device, according to various embodiments. The wearable device includes a band 1701, a pressure actuator 1730, one or more sensors 1731, and circuitry 1711. The one or more sensors 1731 may include a pressure sensor 1732 and a pulse wave velocity (PWV) sensor 1733 in one embodiment, but in other embodiments the one or more sensors 1731 may include any combination of sensors described in any example of blood pressure monitoring described herein (expressly or in those applications incorporated by reference).

The circuitry 1711 may include an actuator control 1721 (also referred to as a “controller” herein) and a processor 1722 to perform any blood pressure calculations described herein (expressly or in those applications incorporated by reference) alone or in combination with a remote processor. The actuator control 1721 may be an application specific machine, such as logic or some other integrated circuit in one embodiment, while the processor 1722 may be a general purpose machine that is transformed into a special purpose machine by loading instructions associated with any blood pressure calculations described herein (expressly or in those applications incorporated by reference). In examples in which the processor 1722 co-operates with a remote processor (not shown), the processor 1722 may transmit raw measurement data or information derived therefrom to the remote processor for remote calculation of the blood pressure value. In examples in which the processor 1722 co-operates with a remote processor, the one of the processors that generates the blood pressure value may be referred to as the “calculator.”

As explained above, the actuator control 1721 may be a component separate from processor 1722, but in other examples the actuator control 1721 may be implemented in the processor 1722. For instance, the actuator control 1721 may be a module executed by the processor 1722, an individual core of the processor 1722 separate from a core used to do blood pressure calculations, or the like, according to various embodiments. The term “circuitry” 1711 is meant to refer to any combination of general purpose machines and/or application specific machines to implement the actuator control 1721 and the processor 1722.

The band 1701 is operable to apply external pressure to a part of a human body and sense the external pressure. The PWV sensor 1733 is operable to sense signals related to pulse wave velocity (PWV). The actuator control 1711 controls components of the wearable device 1701. The circuitry 1711 has a first measurement mode operable to control the actuator 1730 to apply the external pressure, changing only in a certain range less than 80 mmHg. Processor 1711 may be operable to receive a waveform signal of the external pressure (oscillometric waveform) from the pressure sensor 1732 and the signals from the PWV sensor 1733 (PWV signal) to calculate a blood pressure value based on the oscillometric waveform and the PWV signals in the first mode.

In another embodiment, a blood pressure monitoring system 1700 also includes an external device where the collected data from the wearable device 1701 can be sent and stored, and a user-facing application where both the doctor and patient can access the collected data. The wearable device 1701 may include a wireless connection to the external device.

In another embodiment, the external device includes the calculator and the wearable device 1701 does not include the calculator, but instead includes a distributed computing module, such as a transmit module to transmit the data from the band and the PWV sensor to the external device. In these examples, processor 1722 may be a processor to execute this distributed computing module, and the remote processor may be the calculator.

In another embodiment, the PWV sensor 1733 is any one of two photoplethysmography's (PPG), one PPG and one electrocardiograph (ECG), two PPGs and one ECG, two pressure actuators and two pressure sensors, one pressure actuator and one pressure sensor and one ECG, or two pressure actuators and two pressure sensors and ECG.

In another embodiment, the one or more sensors 1731 may include, but are not limited to: oscillometric sensors, pressure sensors, pulse wave velocity (PWV) sensors, photoplethysmography (PPG) sensors with various outputs (for example, infared, radar, red, green or imaging PPGs), electrocardiogram (ECG) sensors, motion sensors, force sensors, pumps, valves, shape memory alloys, transmitters, and air bladders.

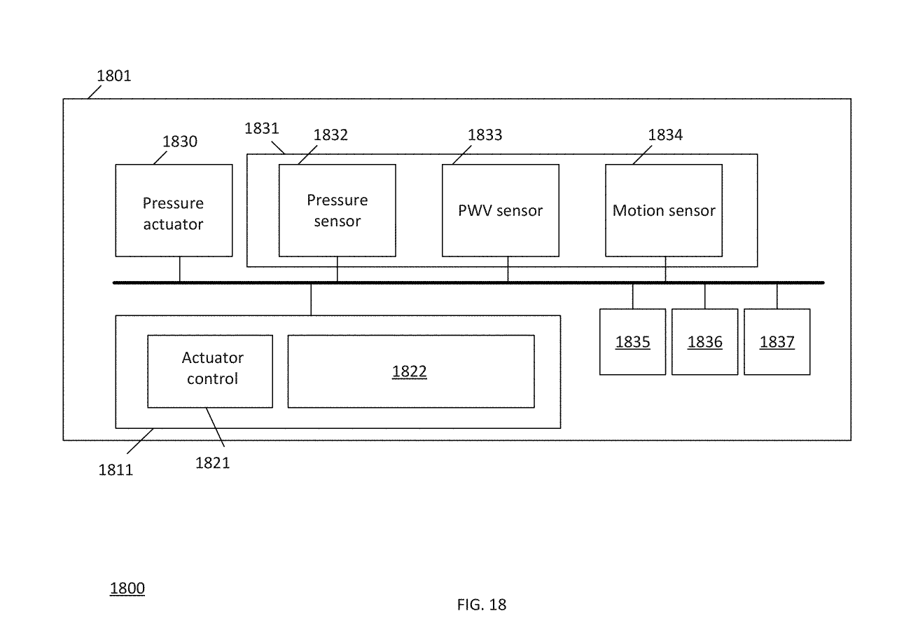

FIG. 18 is a block diagram of a low blood pressure actuation blood monitoring system 1800 in which the wearable device 1801 includes a motion sensor 1834, a user interface 1835 (such as a touch monitor), a memory 1836 to store collected data and/or calculated values, and a wireless I/O 1837, according to various embodiments. Pressure actuator 1830 may be similar to any pressure actuator described herein (such as those described with reference to FIG. 17). One or more sensors 1831, pressure sensor 1832, and PWV sensor 1833, may be similar to any one or more sensors described herein (such as those described with reference to FIG. 17). Circuitry 1811, actuator control 1821, and processor 1822 may be similar to any circuitry, control, and processor described herein (such as those described with reference to FIG. 17). Circuitry 1811 may further use information collected by the motion sensor 1834 to determine which measurement mode to use for a particular reading, as described anywhere herein, particularly in the section with the heading “Usage in Daily Activities.”

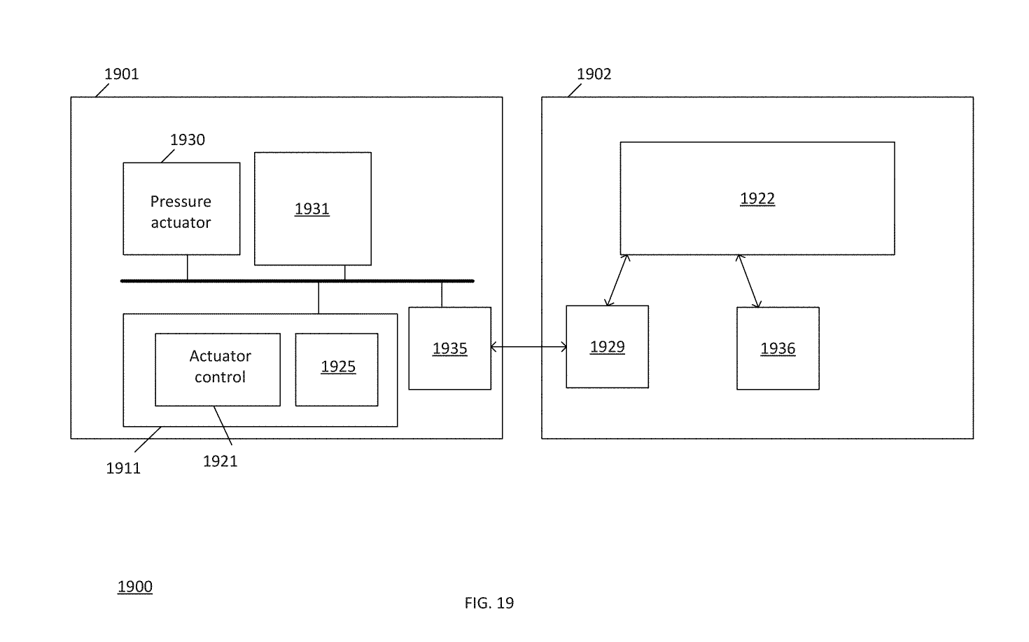

FIG. 19 is a block diagram of a low blood pressure range blood monitoring system 1900 in which the wearable device 1901 wirelessly transmits raw measurement data or information derived therefrom to a cloud device 1902 for remote calculation of blood pressure value, according to various embodiments. Pressure actuator 1930 may be similar to any pressure actuator described herein (such as those described with reference to FIG. 17). One or more sensors 1931 may be similar to any one or more sensors described herein (such as those described with reference to FIG. 17). Actuator control 1921 may be similar to any control described herein (such as those described with reference to FIG. 17).

Processor 1925 of circuitry 1911 may co-operate with processor 1922 of the cloud device 1902 to generate a blood pressure value. For example, processor 1925 may collect data from the sensors 1931 and transmit that data (or information derived therefrom) via wireless module 1935, over an electronic network, for receipt by network module 1929 (which may be any network interface). Processor 1922 may perform any operations of any calculator or processor described herein, such as any operations performed by processor 1722 of FIG. 17. Processor 1922 may store a generated blood pressure value in local storage 1936 or any storage accessible by cloud device 1902 (such as a remote database).

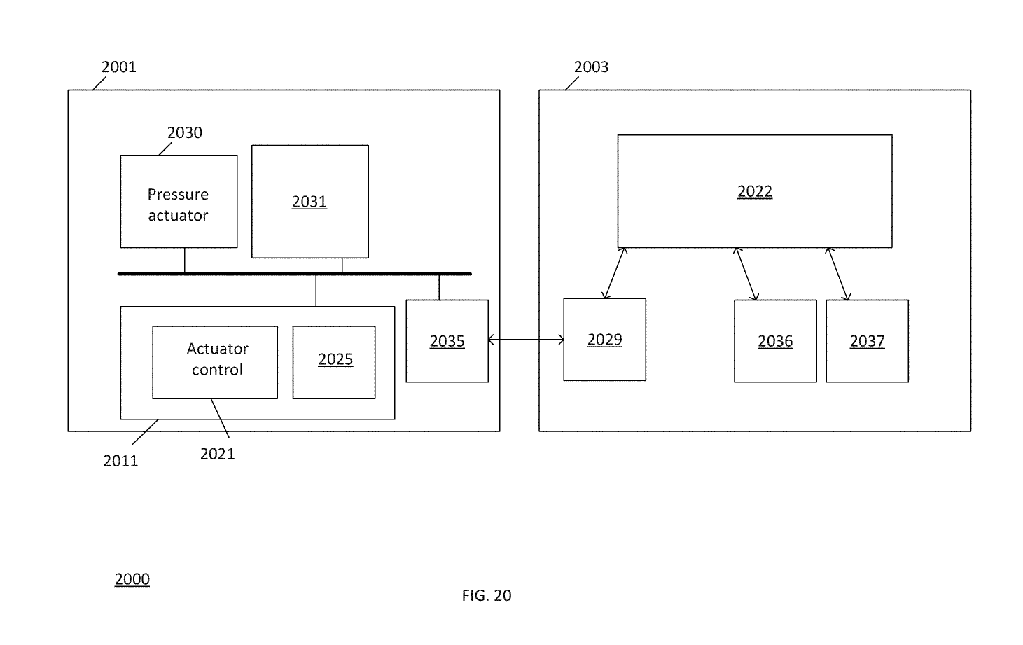

FIG. 20 is a block diagram of a low blood pressure range blood monitoring system 2000 in which the wearable device 2001 transmits raw measurement data or information derived therefrom over a short range wireless connection or a wire to a portable device for calculation of the blood pressure value by the portable device 2003. Pressure actuator 2030 may be similar to any pressure actuator described herein (such as those described with reference to FIG. 17). One or more sensors 2031 may be similar to any one or more sensors described herein (such as those described with reference to FIG. 17). Actuator control 2021 may be similar to any control described herein (such as those described with reference to FIG. 17).

Interface 2035 may be a short range wireless interface (such as Bluetooth) or a wire coupled to a similar interface 2029 on portable device 2003. Processor 2025 may be similar to processor 1925 (FIG. 19) and processor 2022 may be similar to processor 1922 (FIG. 19). Storage 3036 may be similar to storage 1936 (FIG. 19). User interface 2037 may be similar to any user interface described herein, such as interface 1837 in FIG. 18.

Most of the equipment discussed with reference to FIGS. 17-20, for instance the circuitry 1711 (FIG. 17), the circuitry 1811 (FIG. 18), the circuitry 1911 and the processor 1922 (FIG. 19), and the circuitry 2011 and the processor 2022 (FIG. 20), may comprise hardware and associated software. For example, the typical circuitry is likely to include one or more processors and software executable on those processors to carry out the operations described. We use the term software herein in its commonly understood sense to refer to programs or routines (subroutines, objects, plug-ins, etc.), as well as data, usable by a machine or processor. As is well known, computer programs generally comprise instructions that are stored in machine-readable or computer-readable storage media. Some embodiments of the present invention may include executable programs or instructions that are stored in machine-readable or computer-readable storage media, such as a digital memory. We do not imply that a “computer” in the conventional sense is required in any particular embodiment. For example, various processors, embedded or otherwise, may be used in equipment such as the components described herein.

Memory for storing software again is well known. In some embodiments, memory associated with a given processor may be stored in the same physical device as the processor (“on-board” memory); for example, RAM or FLASH memory disposed within an integrated circuit microprocessor or the like. In other examples, the memory comprises an independent device, such as an external disk drive, storage array, or portable FLASH key fob. In such cases, the memory becomes “associated” with the digital processor when the two are operatively coupled together, or in communication with each other, for example by an I/O port, network connection, etc., such that the processor can read a file stored on the memory. Associated memory may be “read only” by design (ROM) or by virtue of permission settings, or not. Other examples include but are not limited to WORM, EPROM, EEPROM, FLASH, etc. Those technologies often are implemented in solid state semiconductor devices. Other memories may comprise moving parts, such as a conventional rotating disk drive. All such memories are “machine readable” or “computer-readable” and may be used to store executable instructions for implementing the functions described herein.

A “software product” refers to a memory device in which a series of executable instructions are stored in a machine-readable form so that a suitable machine or processor, with appropriate access to the software product, can execute the instructions to carry out a process implemented by the instructions. Software products are sometimes used to distribute software. Any type of machine-readable memory, including without limitation those summarized above, may be used to make a software product. That said, it is also known that software can be distributed via electronic transmission (“download”), in which case there typically will be a corresponding software product at the transmitting end of the transmission, or the receiving end, or both.

### Calculation of Blood Pressure

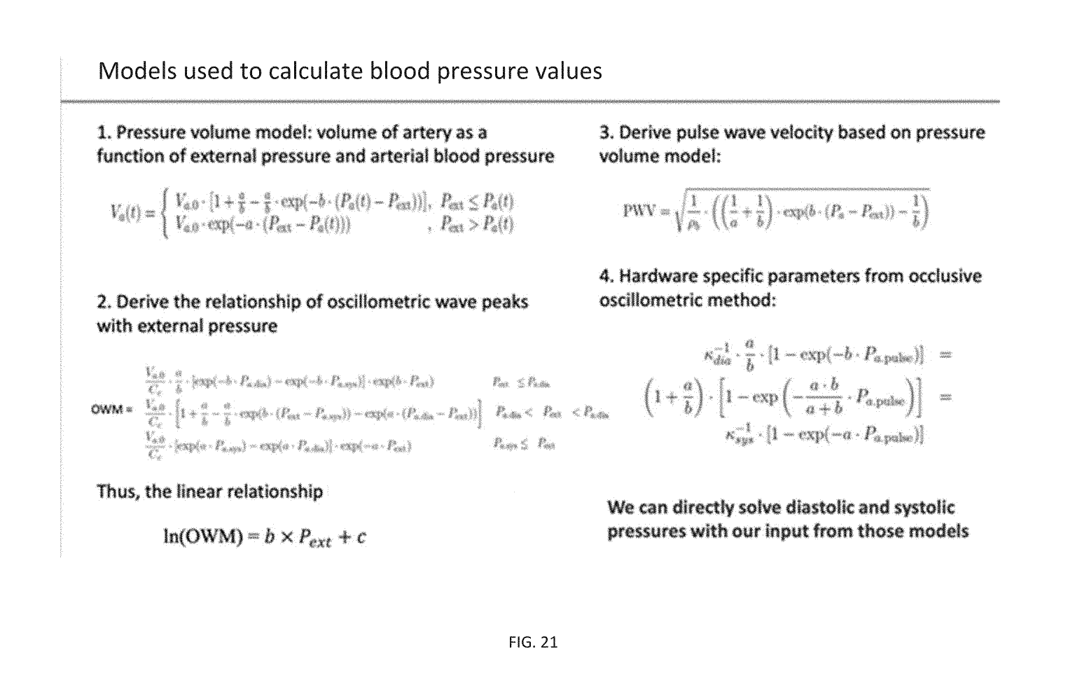

A user wears any wearable device described herein on a part of the body where an artery is present when measuring blood pressure. The wearable device may be configured to perform any operations associated with low pressure actuation blood pressure monitoring described herein. In some examples, any wearable device described herein and/or any calculator described herein may be configured to perform any operations expressly described herein (or otherwise, including any operations described in those applications incorporated by reference), including those described with respect to FIGS. 1-16, particularly FIG. 5 and the description thereof. Also, FIG. 21 is an illustration summarizing earlier described models that may be utilized by any wearable device and/or calculator described herein to calculate a blood pressure value based on a waveform signal from a pressure sensor and signals associated with PWV, according to various embodiments. Any calculator described herein may use any of the models shown in FIG. 21 and/or described elsewhere in the present application (e.g., expressly such as the models described with reference to FIGS. 1-16 or otherwise, including any operations described in those applications incorporated by reference).

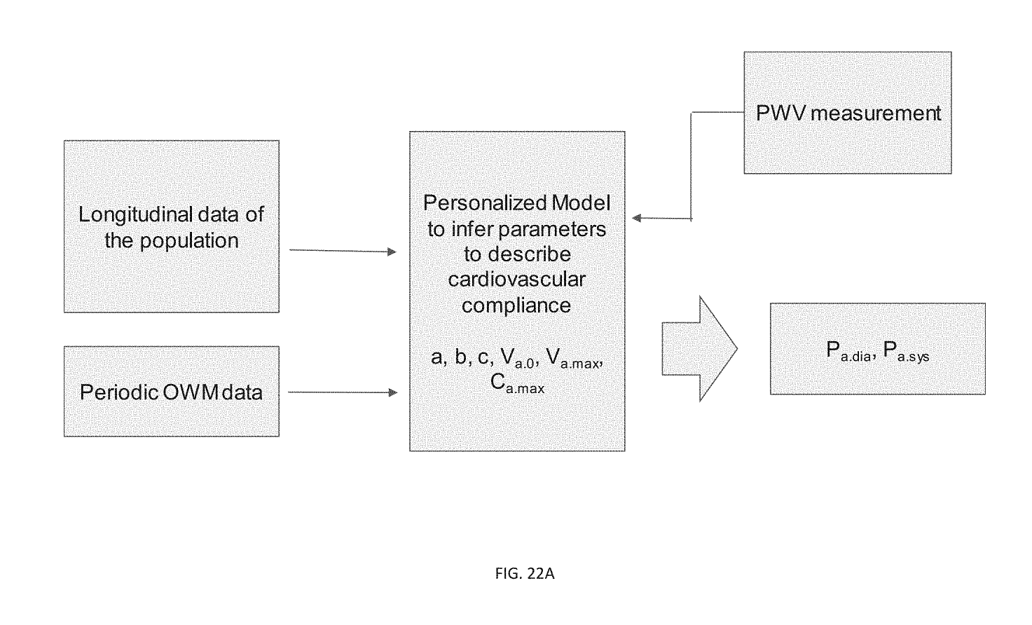

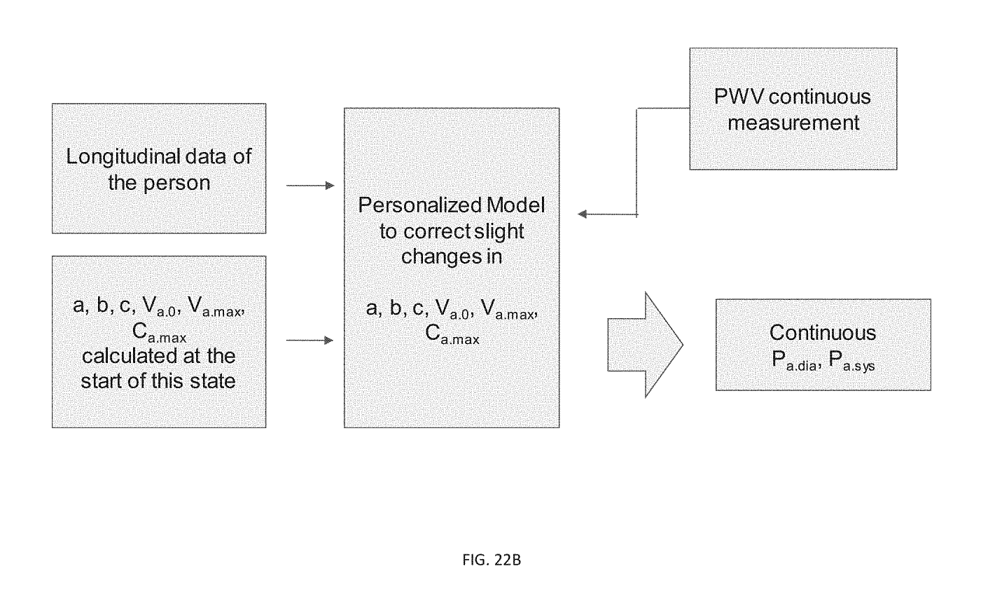

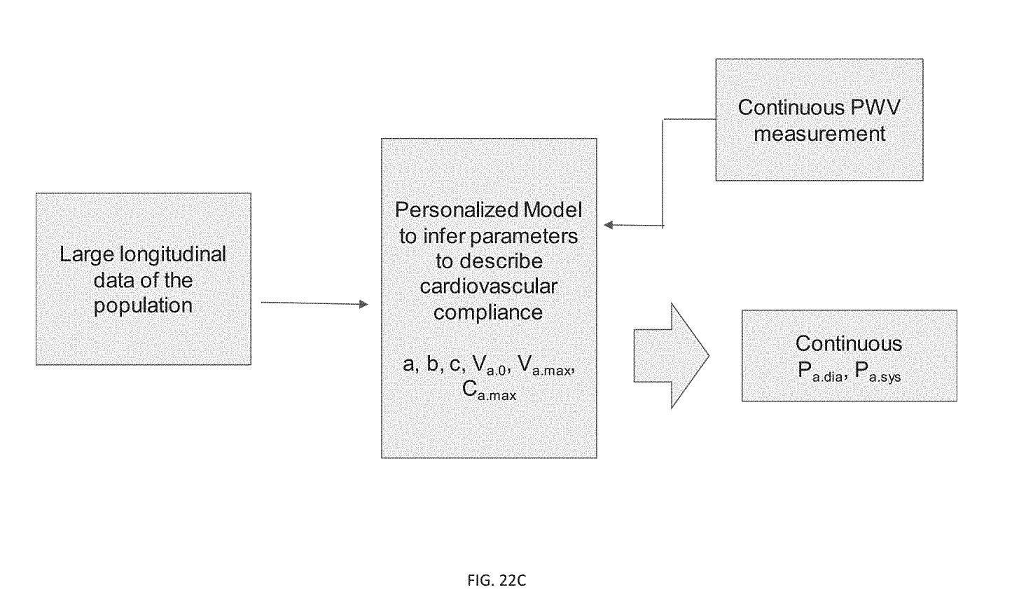

In one embodiment, in the first mode, any calculator described herein may include a precise algorithm designed to accurately calculate the diastolic and systolic blood pressures by using the data collected through low-pressure oscillometry augmented with signals from PWV sensor(s) within the wearable device. In a non-limiting example, the device may first measure the cardiovascular parameters of the user, such as volume of artery, compliance of artery, and changes of volume and compliance, using a series of low-pressure oscillometric waves controlled by the air bladder, pumps, and valves, or with the addition of PPGs and ECGs, to build a model on the relationship of artery volume with regards to the changing transmural pressure on the artery, followed by inputting sensor signals and other relevant information into the model to inference blood pressure values. Using previously inputted hardware-specific parameters, and any patient biometrics (if applicable), the device may evaluate the collected data through the pre-loaded algorithm, and therefore be able to accurately calculate both the diastolic and systolic blood pressures with reduced amount of sensor input than the first example. The accuracy will be improved once the blood pressure monitoring system receives more data from different users or/and repeatedly for the same users. This can be achieved by Bayesian Neural Network, reinforcement learning, transfer learning, and other deep learning methods to generate a personalized model as illustrated in FIG. 22A.

In some embodiments, the system will also take the user's biometric information into account, either as provided by the user at the start of use (FIG. 22B) or as determined by the sensors within the wearable band during use (including, but not limited to, height, weight, age, cholesterol levels, degree of normal activity, etc.). Some embodiments are also customizable for each patient, including, but not limited to, the size of the wearable band, the amount of biometric information inputted at the start of use, which sensors may be primarily used to gather the most accurate data needed for the algorithm, and the frequency in which the device becomes active to take measurements and evaluate blood pressure, depending on the advice of the residing physician (for example, every half hour, every hour, every three hours, etc.). Once the system has obtained longitudinal data of the population, the calculation model is improved to be able to calculate more accurate blood pressure through self-learning of the model parameters, and to calculate blood pressure based on the signal from the PWV sensor(s) without the oscillometric waveforms (FIG. 22C). The controller has a third mode operable to control the calculator to calculate blood pressure based on the signal from the PWV sensor(s), and control the pressure actuator not to work (FIG. 22B).

### Wearable Devices

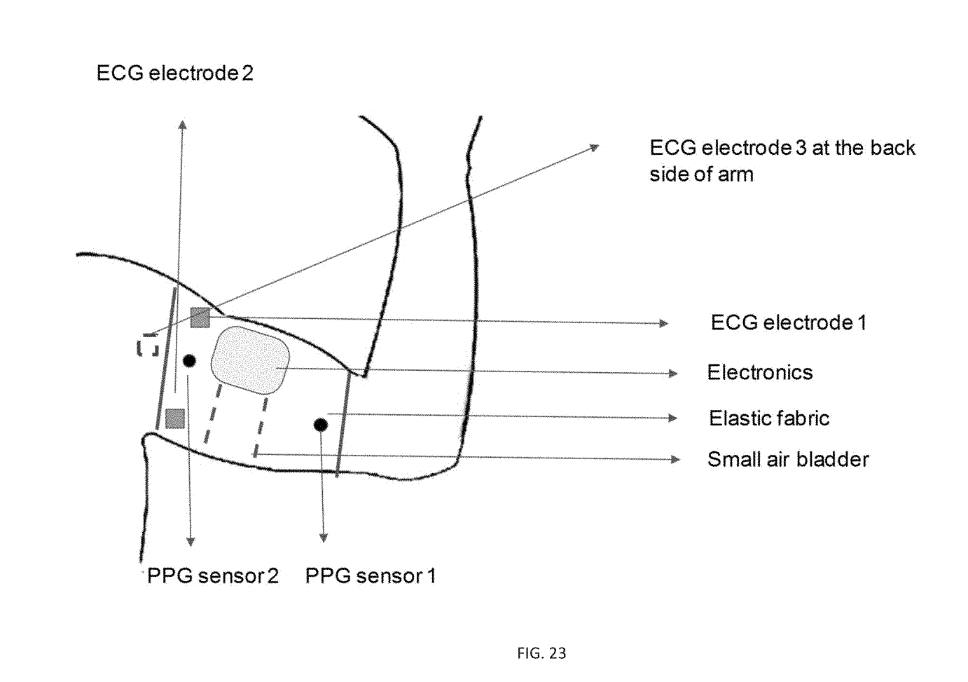

Any wearable device described herein may be designed to be worn on specific parts of the body where an artery is present, including, but not limited to, the upper arm, forearm, wrist, fingers, or thigh. The device can also be divided into several parts, and each sensor applied to different parts of the body for optimal signal quality. In some embodiments, the wearable device is designed to be worn on the left upper arm, where the band can sense best quality oscillometric waveforms because of the simple bone structure of the upper arm, and ECG electrodes on a single arm can generate signals sufficient for building the model due to the short distance to the heart. Such an example is illustrated in FIG. 23.

Measurement of blood pressure is more accurate when the wearable device works on the specified parts. When the wearable device is worn on other parts of the body, for example, the right upper arm, the controller generates and sends an alert to the user. In some embodiments, the controller detects that the wearable device is worn on other parts of the body based on the motion of the device, detected by the motion sensor.

In some embodiments, the wearable device can be worn on any part of the body where an artery is present, as preferred by the user, including, but not limited to, the upper arm, forearm, wrist, or thigh. In some embodiments, the wearable device is able to adjust how it measures blood pressure and collect data based on which part of the body it is worn on to ensure the greatest accuracy. In some embodiments, the hardware within the wearable cuff is able to automatically adjust in real-time if the wearer moves the wearable device from, for example, the upper arm to the thigh. The adaptability of the device allows the patient a greater level of privacy if they wish to cover the wearable device when going out in public because it can be worn under any clothing desired by the user. In some embodiments, the wearable cuff is made from an elastic fabric for optimal comfort to the user during wear.

In some embodiments, the wearable device is able to be worn for twenty-four hours, the standard wear-time for a 24 hour ambulatory blood pressure monitoring device in order to receive the minimum amount of easy-to-digest, useable data. In some embodiments, the wearable device is able to be worn for more or less than 24 hours, if necessary.

### Comfortableness

In any embodiments described herein, in the first mode, the wearable device detects the wearer's blood pressure using only low-pressure oscillometry, or “pulse pressure waveforms”, making the band physically more comfortable for the wearer, particularly long-term. It is observed that most blood pressure measurement devices in the current market, for example, Sphygmomanometers, rely on high pressure oscillometry (essentially squeezing the enclosed area using high pressure to cut off blood flow in the artery, then comparing the oscillation of blood pulsation when the artery is totally collapsed with when the artery is partially collapsed as well as in natural states, and use either sound or algorithms to identify systolic and diastolic pressures), which is not only uncomfortable for the user, even for a single point measurement, but also generally provides inaccurate estimates for systolic and diastolic blood pressure measurements—both of which are necessary for diagnosing and evaluating the treatment for patients with hypertension. In some embodiments, the blood pressure device applies external pressure less than a mean arterial blood pressure of the user, for example, below 100 mmHg, or a diastolic pressure of the user, for example, below 80 or 60 mmHg. In some embodiments, the external pressure changes within a range of 20-80 mmHg, 20-50 mmHG, or 20-40 mmHg, where currently available high-pressure devices may use, for example, a minimum range of 140-300 mmHg of external pressure.

### Higher Pressure Mode

In other embodiments, the controller of the wearable device also has a second mode operable to control the pressure actuation to above the range of operation in the first mode. The band is controlled to apply the external pressure changing the range used in the first mode to above the diastolic pressure, above the mean pressure, or above the systolic pressure, for example, up to 160 mmHg. In this second mode, the device works to generate and collect more ocillometric waveforms than the first mode, and thus more information to extract for inferencing blood pressure. This mode is sometimes useful to compare the results for calibrating the calculation, but is uncomfortable for the wearer. It is better to limit the use of the second mode only as needed in suitable situations. In some embodiments, the controller prohibits the second mode when the motion is less than a certain level. For example, the device can avoid using the second mode when the user wearing the device is sleeping. In another embodiment, the device also includes a command receiver (button, touch panel, voice command input, pre-programmed protocol, etc.) operable to receive a command to start the second mode, and the controller starts the second mode when the receiver receives the command. It is best to avoid frequent usage of the second mode. The user or doctor can start the second mode only as needed.

### Usage in Daily Activities

In some embodiments, the wearable device is able to be worn through a variety of activities, including, but not limited to, walking, running, driving, exercising, reading, and sleeping. In some embodiments, the wearable device is able to automatically identify the activity of the user by using sensor data (i.e. pressure, ECG, PPG, motion sensor), and adjust the use of low-pressure actuation and any additional sensors accordingly to ensure the most accurate reading of the users blood pressure based on, for example, how fast the heart is beating, the volume of the arteries, the compliance of arteries, the velocity of blood flow, the various activities the wearer may be participating in during use, which are all information useful for inferencing the hidden parameters of models for calculating blood pressure. It is observed that due to the reliance on primarily high pressure, conscious patients are unable to wear the current sphygmomanometer devices during their sleep cycle as the intensity of the pressure wakes the patient and therefore deems the reading inaccurate. However, it is also observed that an accurate reading of blood pressure during a patients' sleep cycle is very effective in determining a clear depiction of risk for that patient, as high blood pressure readings during sleep is a sure indicator of hypertension, and likely elevated risk for more serious diseases as a result. This low-pressure invention makes comfortable, non-interruptive blood pressure monitoring during sleep possible. In some embodiments, the wearable device is also quiet when the low-pressure actuation are functioning to read the patient's blood pressure in the first mode, allowing the patient to remain in slumber for both the lack of sudden noise, and lack of sudden, intense pressure where the cuff is being worn.

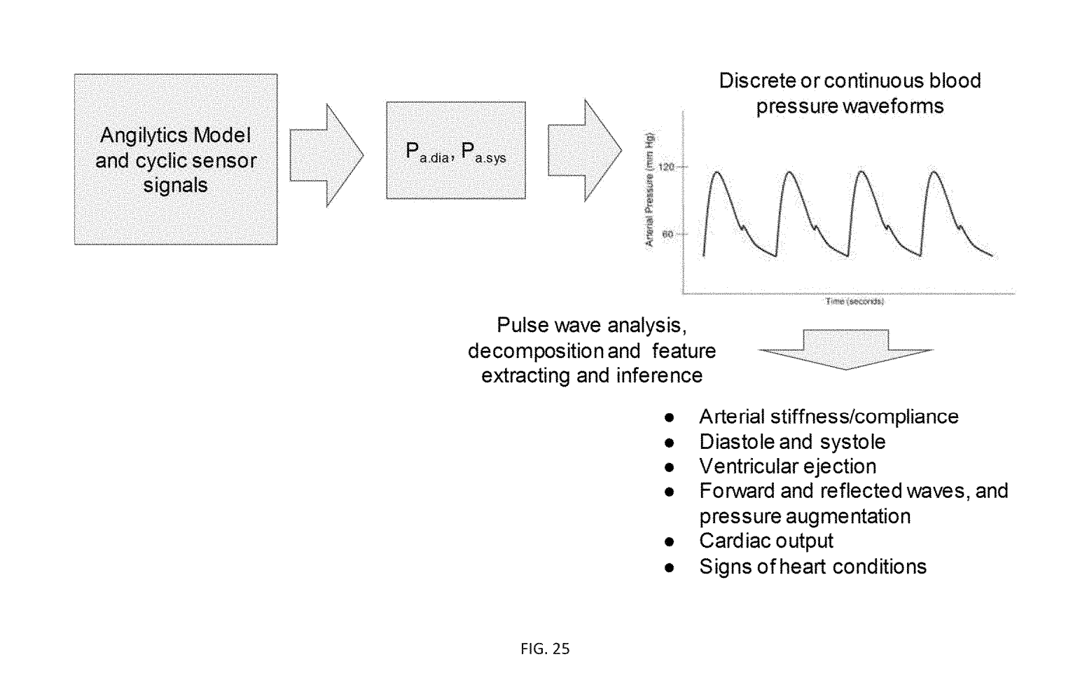

In some embodiments, the wearable device measures blood pressure frequently, for example, every 10, 20, or 30 minutes. In some other embodiments, the wearable device measures blood pressure continuously. Additional information may be derived from these measurements as illustrated in FIG. 25. In some embodiments, the wearable device is programmed to vary the amount of pressure necessary to achieve accurate reading. For example, when the device senses that a user's activity has changed from rest to a medium amount of activity (i.e., the changes in heart beat and blood flow, or change beyond a threshold for motion sensor reading) and thus from a relatively stable state to unstable state where the vascular tone will change and hidden parameters of the model vary, the device may adjust the amount of external pressure, or the frequency of applying external pressure to ensure that the variability as a result of physiological fluctuations has been properly captured to ensure the accuracy of measurement.

### Self-Adapting and Feedback Loop

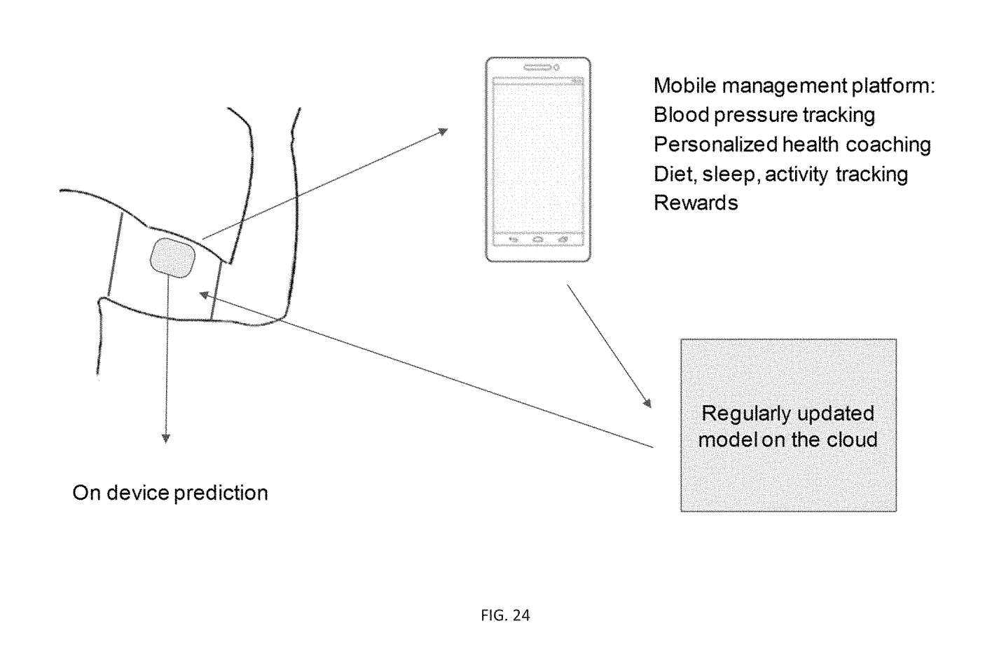

In some embodiments, because the device is able to adapt based on the physical circumstances of the wearer, it may also be programmed to adapt itself after each reading to ensure its ability to re-sense the correct amount of pressure and the maximum pressure necessary at each reading. By including a capability to adapt the amount of external pressure used at each reading and the frequency of applying pressure for measurements, the device minimizes the need for pressure actuation for power conservation and maximum comfort and wearability. In some embodiments, the self-adapting at each reading may also allow the blood pressure monitoring system to utilize a feedback loop, where previously collected data can be ingested and applied to the following readings for even greater accuracy for that patient. In some embodiments, this may also be completed with the assistance of machine learning and deep learning attributes, allowing the blood pressure monitoring system to also process these data insights with additional accuracy and efficiency, meaning the blood pressure monitoring system has the ability to grow smarter the more it is used, and may therefore require less frequent readings while still maintaining a high level of accuracy, as described throughout the present application such as in FIGS. 12A-B and the description thereof. FIG. 24 illustrates one example of a system utilizing a feedback loop.

### Applications

Some embodiments of the blood pressure monitoring system may also include a corresponding application where the collected data and/or measured data is stored and displayed in a user-friendly interface, which can be loaded onto an external device such as, but not limited to, a smartphone, tablet, or display monitor included in the wearable device. In some embodiments, both the patient using the device and the prescribing physician are able to access the application, with user-specific preferences, in order to review output from the wearable device or results from calculator. In some embodiments, the corresponding blood pressure monitoring application will also encourage the patient to engage with their own health information by incorporating additional aspects, such as, but not limited to, a medication tracker, activity tracker, biometric surveys, diet tracker, and important data outputs translated into a user-friendly, more understandable explanation. In a non-limiting example, a high-risk hypertension patient utilizing the wearable device may receive a real-time message in their application warning them that their blood pressure has spiked within a given timeframe, instead of a table of the numerical readings that the device has measured from the wearable device during that timeframe. In some embodiments, messages which contain warning information, such as listed in the above example, may also include a real-time alert to the device where the application has been loaded, i.e. smartphone or tablet. Some embodiments also support messages in the application that are not real-time as dependent on the content. Some embodiments also include an identical real-time alert sent to the residing physician, so that they may immediately check in with the patient and/or suggest emergency medical care as needed. In other embodiments, user may program the application to send the warning message to his/her doctor after he/she has received the message. In some embodiments, the application may also be able to predict the specific disease risk for the patient using the device based on the pre-loaded parameters (as described above), and the real-time blood pressure reading(s), in particular where machine learning attributes are utilized. In these embodiments, this information may be displayed in one or both of the patient and physician applications.

### Other Utilization of Data

In some embodiments, with patient consent, all or some of the data collected and interpreted by the blood pressure monitoring system can be utilized by the residing facilities in order to gain insights, for future and current hypertension patient care, thereby increasing preventative care information and measures, and reducing the amount of hypertension-related diseases and deaths. In a non-limiting example, signals may provide additional insights in patients with similar hypertension readings and biometrics to help healthcare providers better evaluate and stratify risks of patients, prescribing personalized medication, participating in targeted interventions, or additional blood pressure management. In some embodiments, the combination of hardware and software utilized in the blood pressure monitoring system may be reconfigured for countless other medical-monitoring applications, such as, but not limited to, heart functionalities, patient interactions with new medications, cardiovascular health, breathing, brain activity, rehabilitation and trauma management, and anxiety and other mental health issues that cause physiological changes in the patient, etc. In some embodiments, the materials and hardware utilized in the construction of some of the blood pressure monitoring systems described herein are inexpensive to build, and are therefore inexpensive for potential users to either rent or purchase, either in partnership with a hospital or payers, or as a consumer goods for those who want to self-monitor. This may provide early prevention from developing into something more serious due to unmanaged hypertension, as well as cost savings of healthcare expenditure currently related to uncontrolled hypertension.

It will be obvious to those having skill in the art that many changes may be made to the details of the above-described embodiments without departing from the underlying principles of the invention. The scope of the present invention should, therefore, be determined only by the following claims.

Having described and illustrated the principles of the invention in a preferred embodiment thereof, it should be apparent that the invention may be modified in arrangement and detail without departing from such principles.

1. A system, comprising:
a band;
a pressure actuator to apply external pressure through the band to a part of a human body;
circuitry to control the pressure actuator to apply the external pressure changing only in a pressure range less than 80 mmHg in a measurement mode; and
a pressure sensor to sense, from the band, a waveform signal responsive to an application of the external pressure by the pressure actuator in the measurement mode, wherein the waveform signal is indicative of a pressure response of arterial pressure;
a pulse wave velocity (PWV) sensor to sense one or more signals associated with PWV;
the circuitry further including a memory and one or more processors, the memory having instructions stored thereon that, in response to execution by the one or more processors, cause the one or more processors to perform operations comprising:
locally calculating blood pressure value based on the waveform signal from the pressure sensor and the signal(s) associated with PWV, or
transmitting the waveform signal and the signal(s) associated with PWV, or information derived therefrom, to a remote processor for remote calculation of the blood pressure value based on the waveform signal from the pressure sensor and the signal(s) associated with PWV.

2. The system of claim 1, wherein the pressure range is less than 50 mmHg.

3. The system of claim 1, wherein the pressure range is at least 20 mmHg.

4. The system of claim 1, wherein the measurement mode comprises a first measurement mode, the external pressure comprises a first external pressure, and the pressure range comprises a first pressure range, and wherein the circuitry is further to:
control the pressure actuator to apply a second external pressure in a second pressure range in a second measurement mode, wherein the second pressure range includes a maximum pressure that is greater than a maximum pressure of the first pressure range.

5. The system of claim 1, wherein the circuitry is further to, after a local or remote calculation of the blood pressure value, control the PWV sensor to obtain one or more additional signals associated with PWV in an operational state in which the pressure actuator is not used, and the operations further comprise:
locally calculating an additional blood pressure value based on the additional signal(s) associated with PWV, or
transmitting the additional signal associated with PWV, or information derived therefrom, to a remote processor for remote calculation of the additional blood pressure value based on the waveform signal and the additional signal associated with PWV.

6. The system of claim 5, wherein the waveform signal comprises a first waveform signal, and wherein the operations further comprise:
locally inferring a second waveform signal using the additional signal(s) associated with PWV, wherein the local calculation of the additional blood pressure value is based on the additional signal(s) associated with PWV and the second waveform signal, or
transmitting the additional signal(s) associated with PWV to the remote processor for remote inference of the second waveform signal using the additional signal(s) associated with PWV, wherein the remote calculation of the additional blood pressure value is based on the additional signal(s) associated with PWV and the second waveform signal.

7. The system of claim 5, further comprising:
a motion sensor to detect a motion of the band;
wherein the second measurement mode is activated in response to motion less than the threshold.

8. The system of claim 1, wherein the PWV sensor comprises:
a first photoplethysmography sensor (PPG) and at least one of:
a electrocardiograph sensor (ECG), or
a second PPG.

9. The system of claim 8, wherein the PWV sensor comprises the first PPG, the second PPG, and the ECG.

10. The system of claim 5, wherein the operations further comprise entering the operational state based on information collected from a motion sensor or existing knowledge of user subjects.

11. The system of claim 1, wherein the operations further comprise recording the blood pressure value in a local storage or transmitting the blood pressure value over an electronic network for remote storage.

12. The system of claim 1, wherein the operations further comprise recording a time value indicative of measurement time for the blood pressure value in a local storage or transmitting the time value over an electronic network for remote storage.

13. The system of claim 5, wherein the operations further comprise recording, in a local storage, the additional blood pressure value and a value indicative of motion measured at measurement time for the additional blood pressure value, or transmitting the additional blood pressure value and the value indicative of the motion over an electronic network for remote storage.

14. The system of claim 13, wherein:
the value indicative of the motion measured at the measurement time for the additional blood pressure value comprises raw information from a motion sensor or information derived from the raw information from the motion sensor; or
the value indicative of the motion measured at the measurement time for the additional blood pressure value is indicative of magnitude of movement or estimated behavior of the user.

15. The system of claim 1, wherein the operations further comprise:
sensing whether the band is worn on the human body the sensors or an additional sensor; and
prohibiting operation of the pressure actuator when the band is not worn on the human body, or
recording values indicating times that the band is or is not worn on the human body in a local storage or transmitting the values over an electronic network for remote storage.

16. The system of claim 1, wherein the band is wearable on a predetermined part of the human body, and wherein the operations further comprise:
detecting if the band is worn on a part of the human body that is not the predetermined part of the human body; and
generating an alert if the band is worn on the part of the human body that is not the predetermined part of the human body.

17. The system of claim 16, wherein the operations further comprise detecting the band worn on the part of the human body that is not the predetermined part of the human body based on generating a profile using a motion sensor of the system and comparing a measured value to the profile.

18. The system of claim 1, wherein the operations further comprise controlling the pressure actuator to apply the external pressure changing only in a pressure range less than 80 mmHg in a measurement mode.

19. A system, comprising:
a band;
a pressure actuator to apply external pressure through the band to a part of a human body;
circuitry to control the pressure actuator to apply the external pressure changing only in a pressure range less than 80 mmHg in a measurement mode; and
a first sensor to sense, from the band, a waveform signal responsive to an application of the external pressure by the pressure actuator in the measurement mode, wherein the waveform signal is indicative of a pressure response of arterial pressure;
the circuitry further including a memory and one or more processors, the memory having instructions stored thereon that, in response to execution by the one or more processors, cause the one or more processors to perform operations comprising:
locally calculating blood pressure value based on the waveform signal from the pressure sensor and additional information based on a current or previous reading, or
transmitting the waveform signal, or information derived therefrom, to a remote processor for remote calculation of the blood pressure value based on the waveform signal from the pressure sensor and the additional information based on a current or previous reading.

20. The system of claim 19, wherein:
the additional information is based on the current reading and includes time variation information based on blood flow through a blood vessel or one or more signals associated with pulse wave velocity (PWV), wherein the current reading is obtained using at least one photoplethysmography sensor (PPG) and at least one electrocardiograph sensor (ECG), two or more PPGs, without any PPG by using the pressure sensor and one or more ECGs, without any PPG or ECG using the pressure sensor and an additional pressure sensor; or
the additional information is based on the previous reading and includes information from a one-time calibration with a blood pressure device or information from periodic application of pressure using a pressure greater than said external pressure.
